(19) United States
(12) Patent Application Publication
(10) Pub. No.: US 2024/0383992 A1
(43) Pub. Date: Nov. 21, 2024

(54) BCMA-TARGETING CHIMERIC ANTIGEN RECEPTOR AND USE THEREOF

(71) Applicant: ORICELL THERAPEUTICS CO., LTD., Shanghai (CN)

(72) Inventors: Siye CHEN, Shanghai (CN); Yanhong XU, Shanghai (CN); Hao GUO, Shanghai (CN); Yue YANG, Shanghai (CN); Huijiao LI, Shanghai (CN); Zhifeng XU, Shanghai (CN); Yanan SUN, Shanghai (CN); Huanfeng YANG, Shanghai (CN); Xiaowen HE, Shanghai (CN)

(73) Assignee: ORICELL THERAPEUTICS CO., LTD., Shanghai (CN)

(21) Appl. No.: 18/557,494

(22) PCT Filed: Apr. 26, 2022
(86) PCT No.: PCT/CN2022/089287

§ 371 Date: Oct. 26, 2023

(30) Foreign Application Priority Data
Apr. 27, 2021 (CN) 202110462389.1

(51) Int. Cl. C07K 16/28; C07K 14/705; C07K 14/725; C12N 5/0783

(52) U.S. Cl. CPC C07K 16/2878; C07K 14/7051; C07K 14/70517; C07K 14/70521; C07K 14/70578; C12N 5/0636; C07K 2317/622; C07K 2319/02; C07K 2319/03

(57) ABSTRACT

A chimeric antigen receptor (CAR), containing a BCMA (B cell maturation antigen) binding domain. The BCMA binding domain includes an antibody or an antigen-binding fragment of an antibody that specifically binds to BCMA. The antibody contains a heavy-chain complementarity determining region 3 (HCDR3), a heavy-chain complementarity determining region 2 (HCDR2), and a heavy-chain complementarity determining region 1 (HCDR1). The HCDR3 contains an amino acid sequence as set forth in SEQ ID NO: 25, the HCDR2 contains an amino acid sequence as set forth in SEQ ID NO: 41, and the HCDR1 contains an amino acid sequence as set forth in SEQ ID NO: 21.

### FIELD OF THE INVENTION

This application relates to the field of biomedicine, and in particular to a chimeric antigen receptor targeting BCMA and a use thereof.

### BACKGROUND OF THE INVENTION

BCMA (B cell maturation antigen) mainly regulates the proliferation and survival of B cells, as well as the mature differentiation of B cells into plasma cells (PCs). During the differentiation process to PCs, BCMA molecules are gradually induced to express. Since BCMA molecules are only expressed on the surface of PCs and plasmablast membranes and are not expressed on most B cells, hematopoietic stem cells, and other normal tissues, BCMA molecules have become one of the most ideal target molecules for treating MM.

Chimeric antigen receptor T (CAR-T) cell therapy is a precision targeted therapy that has emerged in recent years and is currently one of the hottest research directions in the field of tumor immunotherapy. The basic principle of this therapy is to transform T lymphocytes through genetic engineering technology into CAR-T cells with specific recognition capabilities, which are expanded and cultured in vitro and then reinfused into the patient's body to attack and kill specific tumor cells. CAR-T cell therapy can be applied to BCMA-targeted tumor immunotherapy.

However, there is no any CAR-T product targeting BCMA at present. In view of BCMA's effectiveness as a therapeutic target in B-cell malignancies, especially in multiple myeloma, there is an urgent need to develop new cell therapies in the field to achieve therapeutic goals by acting on BCMA.

### SUMMARY OF THE INVENTION

This application provides a chimeric antigen receptor (CAR) targeting BCMA. The CAR of this application can be stably and efficiently expressed on the surface of immune effector cells (for example, T cells). The immune effector cells including the CAR have one or more of the following properties: (1) killing BCMA-positive target cells with high killing toxicity, (2) secreting a large amount of cytokines under stimulation of target cells, (3) high targeted proliferation ability, and (4) inhibiting tumor growth and eliminating tumors.

In one aspect, this application provides a chimeric antigen receptor (CAR), comprising a BCMA binding domain, and said BCMA binding domain comprises an antibody that specifically binds to BCMA or an antigen-binding fragment of the antibody, wherein said antibody comprises a light-chain complementarity determining region 3 (HCDR3), and said HCDR3 comprises an amino acid sequence as set forth in SEQ ID NO: 25.

In certain embodiments, said antibody that specifically binds to BCMA comprises a light-chain complementarity determining region 2 (HCDR2), and said HCDR2 comprises an amino acid sequence as set forth in SEQ ID NO: 41.

In certain embodiments, said HCDR2 of said antibody that specifically binds to BCMA comprises an amino acid sequence as set forth in any one of SEQ ID NOs: 22-24.

In certain embodiments, said antibody that specifically binds to BCMA comprises a light-chain complementarity determining region 1 (HCDR1), and said HCDR1 comprises an amino acid sequence as set forth in SEQ ID NO: 21.

In certain embodiments, said antibody that specifically binds to BCMA comprises an HCDR1, an HCDR2, and an HCDR3, wherein said HCDR1 comprises an amino acid sequence as set forth in SEQ ID NO: 21, said HCDR2 comprises an amino acid sequence as set forth in SEQ ID NO: 41, and said HCDR3 comprises an amino acid sequence as set forth in SEQ ID NO: 25.

In certain embodiments, said antibody that specifically binds to BCMA comprises an HCDR1, an HCDR2, and an HCDR3, wherein said HCDR1 comprises an amino acid sequence as set forth in SEQ ID NO: 21, said HCDR2 comprises an amino acid sequence as set forth in any one of SEQ ID NOs: 22-24, and said HCDR3 comprises an amino acid sequence as set forth in SEQ ID NO: 25.

In certain embodiments, said antibody that specifically binds to BCMA comprises any set of HCDR1, HCDR2, and HCDR3, selected from the groups consisting of: (1) HCDR1: SEQ ID NO: 21, HCDR2: SEQ ID NO: 22, and HCDR3: SEQ ID NO: 25; (2) HCDR1: SEQ ID NO: 21, HCDR2: SEQ ID NO: 23, and HCDR3: SEQ ID NO: 25; and (3) HCDR1: SEQ ID NO: 21, HCDR2: SEQ ID NO: 24, and HCDR3: SEQ ID NO: 25.

In certain embodiments, said antibody that specifically binds to BCMA comprises a light chain variable region (VH), said VH comprises a framework region H-FR1, a C terminal of said H-FR1 is directly or indirectly linked to an N terminal of said HCDR1, and said H-FR1 comprises an amino acid sequence as set forth in SEQ ID NO: 42.

In certain embodiments, said H-FR1 of said antibody that specifically binds to BCMA comprises an amino acid sequence as set forth in any one of SEQ ID NOs: 26-29.

In certain embodiments, said VH of said antibody that specifically binds to BCMA comprises a framework region H-FR2, and said H-FR2 is located between said HCDR1 and said HCDR2 and comprises an amino acid sequence as set forth in SEQ ID NO: 43.

In certain embodiments, said H-FR2 of said antibody that specifically binds to BCMA comprises an amino acid sequence as set forth in any one of SEQ ID NOs: 30-32.

In certain embodiments, said VH of said antibody that specifically binds to BCMA comprises a framework region H-FR3, and said H-FR3 is located between said HCDR2 and said HCDR3 and comprises an amino acid sequence as set forth in SEQ ID NO: 44.

In certain embodiments, said H-FR3 of said antibody that specifically binds to BCMA comprises an amino acid sequence as set forth in any one of SEQ ID NOs: 33-36.

In certain embodiments, said VH of said antibody that specifically binds to BCMA comprises a framework region H-FR4, an N terminal of said H-FR4 is linked to a C terminal of said HCDR3, and said H-FR4 comprises an amino acid sequence as set forth in SEQ ID NO: 37.

In certain embodiments, said antibody that specifically binds to BCMA comprises an H-FR1, an H-FR2, an H-FR3, and an H-FR4, wherein said H-FR1 comprises an amino acid sequence as set forth in SEQ ID NO: 42, said H-FR2 comprises an amino acid sequence as set forth in SEQ ID NO: 43, said H-FR3 comprises an amino acid sequence as set forth in SEQ ID NO: 44, and said H-FR4 comprises an amino acid sequence as set forth in SEQ ID NO: 37.

In certain embodiments, in said antibody that specifically binds to BCMA, said H-FR1 comprises an amino acid sequence as set forth in any one of SEQ ID NOs: 26-29, said H-FR2 comprises an amino acid sequence as set forth in any one of SEQ ID NOs: 30-32, said H-FR3 comprises an amino acid sequence as set forth in any one of SEQ ID NOs: 33-36, and said H-FR4 comprises an amino acid sequence as set forth in SEQ ID NO: 37.

In certain embodiments, said antibody that specifically binds to BCMA comprises any set of H-FR1, H-FR2, H-FR3, and H-FR4, selected from the groups consisting of: (1) H-FR1: SEQ ID NO: 26, H-FR2: SEQ ID NO: 30, H-FR3: SEQ ID NO: 33, and H-FR4: SEQ ID NO: 37; (2) H-FR1: SEQ ID NO: 27, H-FR2: SEQ ID NO: 31, H-FR3: SEQ ID NO: 34, and H-FR4: SEQ ID NO: 37; (3) H-FR1: SEQ ID NO: 28, H-FR2: SEQ ID NO: 32, H-FR3: SEQ ID NO: 35, and H-FR4: SEQ ID NO: 37; and (4) H-FR1: SEQ ID NO: 29, H-FR2: SEQ ID NO: 30, H-FR3: SEQ ID NO: 36, and H-FR4: SEQ ID NO: 37.

In certain embodiments, said antibody that specifically binds to BCMA comprises a light chain variable region (VH), and said VH comprises an amino acid sequence as set forth in SEQ ID NO: 46.

In certain embodiments, said VH of said antibody that specifically binds to BCMA comprises an amino acid sequence as set forth in any one of SEQ ID NOs: 5-9.

In certain embodiments, said antibody that specifically binds to BCMA comprises a light-chain complementarity determining region 3 (LCDR3), and said LCDR3 comprises an amino acid sequence as set forth in SEQ ID NO: 40.

In certain embodiments, said LCDR3 of said antibody that specifically binds to BCMA comprises an amino acid sequence as set forth in any one of SEQ ID NOs: 15-16.

In certain embodiments, said antibody that specifically binds to BCMA comprises a light-chain complementarity determining region 2 (LCDR2), and said LCDR2 comprises an amino acid sequence as set forth in SEQ ID NO: 39.

In certain embodiments, said LCDR2 of said antibody that specifically binds to BCMA comprises an amino acid sequence as set forth in any one of SEQ ID NOs: 13-14.

In certain embodiments, said antibody that specifically binds to BCMA comprises a light-chain complementarity determining region 1 (LCDR1), and said LCDR1 comprises an amino acid sequence as set forth in SEQ ID NO: 38.

In certain embodiments, said LCDR1 of said antibody that specifically binds to BCMA comprises an amino acid sequence as set forth in any one of SEQ ID NOs: 10-12.

In certain embodiments, said antibody that specifically binds to BCMA comprises an LCDR1, an LCDR2, and an LCDR3, wherein said LCDR1 comprises an amino acid sequence as set forth in SEQ ID NO: 38, said LCDR2 comprises an amino acid sequence as set forth in SEQ ID NO: 39, and said LCDR3 comprises an amino acid sequence as set forth in SEQ ID NO: 40.

In certain embodiments, said antibody that specifically binds to BCMA comprises an LCDR1, an LCDR2, and an LCDR3, wherein said LCDR1 comprises an amino acid sequence as set forth in any one of SEQ ID NOs: 10-12, said LCDR2 comprises an amino acid sequence as set forth in any one of SEQ ID NO: 13-14, and said LCDR3 comprises an amino acid sequence as set forth in any one of SEQ ID NO: 15-16.

In certain embodiments, said antibody specifically binding to BCMA comprises LCDR1, LCDR2, and LCDR3, and said that specifically binds to BCMA comprises any set of LCDR1, LCDR2, and LCDR3 comprises any set of amino acid sequence, selected from the groups consisting of: (1) LCDR1: SEQ ID NO: 10, LCDR2: SEQ ID NO: 13, and LCDR3: SEQ ID NO: 15; (2) LCDR1: SEQ ID NO: 11, LCDR2: SEQ ID NO: 13, and LCDR3: SEQ ID NO: 16; (3) LCDR1: SEQ ID NO: 12, LCDR2: SEQ ID NO: 13, and LCDR3: SEQ ID NO: 16; and (4) LCDR1: SEQ ID NO: 12, LCDR2: SEQ ID NO: 14, and LCDR3: SEQ ID NO: 16.

In certain embodiments, said antibody that specifically binds to BCMA comprises a light chain variable region (VL), said VL comprises a framework region L-FR1, a C terminal of said L-FR1 is directly or indirectly linked to an N terminal of said LCDR1, and said L-FR1 comprises an amino acid sequence as set forth in SEQ ID NO: 17.

In certain embodiments, said VL of said antibody that specifically binds to BCMA comprises a framework region L-FR2, and said L-FR2 is located between said LCDR1 and said LCDR2 and comprises an amino acid sequence as set forth in SEQ ID NO: 18.

In certain embodiments, said VL of said antibody that specifically binds to BCMA comprises a framework region L-FR3, and said L-FR3 is located between said LCDR2 and said LCDR3 and comprises an amino acid sequence as set forth in SEQ ID NO: 19.

In certain embodiments, said VL of said antibody that specifically binds to BCMA comprises a framework region L-FR4, an N terminal of said L-FR4 is linked to a C terminal of said LCDR3, and said L-FR4 comprises an amino acid sequence as set forth in SEQ ID NO: 20.

In certain embodiments, said antibody that specifically binds to BCMA comprises an L-FR1, an L-FR2, an L-FR3, and an L-FR4, wherein said L-FR1 comprises an amino acid sequence as set forth in SEQ ID NO: 17, said L-FR2 comprises an amino acid sequence as set forth in SEQ ID NO: 18, said L-FR3 comprises an amino acid sequence as set forth in SEQ ID NO: 19, and said L-FR4 comprises an amino acid sequence as set forth in SEQ ID NO: 20.

In certain embodiments, said antibody that specifically binds to BCMA comprises a light chain variable region (VL), and said VL comprises an amino acid sequence as set forth in SEQ ID NO: 45.

In certain embodiments, said VL of said antibody that specifically binds to BCMA comprises an amino acid sequence as set forth in any one of SEQ ID NOs: 1-4.

In certain embodiments, said antibody that specifically binds to BCMA comprises a VH and a VL, said VH comprises an amino acid sequence as set forth in SEQ ID NO: 46, and said VL comprises an amino acid sequence as set forth in SEQ ID NO: 45.

In certain embodiments, said antibody that specifically binds to BCMA comprises a VH and a VL, said VH comprises an amino acid sequence as set forth in any one of SEQ ID NOs: 5-9, and said VL comprises an amino acid sequence as set forth in any one of SEQ ID NOs: 1-4.

In certain embodiments, said antibody that specifically binds to BCMA comprises VH and VL, and said VH and VL comprises any set of amino acid sequence, selected from the groups consisting of: (1) VH: SEQ ID NO: 5 and VL: SEQ ID NO: 1; (2) VH: SEQ ID NO: 6 and VL: SEQ ID NO: 2; (3) VH: SEQ ID NO: 7 and VL: SEQ ID NO: 2; (4) VH: SEQ ID NO: 8 and VL: SEQ ID NO: 2; (5) VH: SEQ ID NO: 9 and VL: SEQ ID NO: 3; (6) VH: SEQ ID NO: 8 and VL: SEQ ID NO: 3; (7) VH: SEQ ID NO: 9 and VL: SEQ ID NO: 4; and (8) VH: SEQ ID NO: 10 and VL: SEQ ID NO: 4.

In certain embodiments, said BCMA binding domain comprises a scFv, and said scFv comprises said VH and said VL of said antibody that specifically binds to BCMA.

In certain embodiments, in said scFv, the C terminal of said VH and the N terminal of said VL are directly or indirectly linked.

In certain embodiments, in said scFv, the C terminal of said VL and the N terminal of said VL are directly or indirectly linked.

In certain embodiments, said scFv comprises a linker peptide between said VH and said VL, and said linker peptide comprises an amino acid sequence as set forth in SEQ ID NO: 58.

In certain embodiments, said scFv comprises an amino acid sequence as set forth in any one of SEQ ID NOs: 49-56.

In certain embodiments, the CAR comprises a transmembrane domain, and said transmembrane domain comprises transmembrane domains derived from proteins selected from the group consisting of: CD28, CD3e, CD45, CD4, CD5, CD8, CD9, CD16, CD22, CD33, CD37, CD64, CD80, CD86, CD134, CD137, and CD154. In certain embodiments, said transmembrane domain is derived from CD8, and said transmembrane domain comprises an amino acid sequence as set forth in SEQ ID NO: 61.

In certain embodiments, the CAR comprises a costimulatory domain, and said costimulatory domain comprises costimulatory domains derived from proteins selected from the group consisting of: CD137, CD28, 4-1BB, OX-40, and ICOS. In certain embodiments, said costimulatory domain is derived from CD137, and said costimulatory domain comprises an amino acid sequence as set forth in SEQ ID NO: 62.

In certain embodiments, the CAR comprises an intracellular signaling domain, and said intracellular signaling domain comprises an intracellular signaling domain derived from CD3ζ. In certain embodiments, said intracellular signaling domain comprises an amino acid sequence as set forth in SEQ ID NO: 63.

In certain embodiments, the CAR comprises a hinge region, and said hinge region comprises a hinge region derived from CD8. In certain embodiments, said hinge region is derived from CD8 and comprises an amino acid sequence as set forth in SEQ ID NO: 60.

In certain embodiments, the CAR comprises a signal peptide. In certain embodiments, said signal peptide comprises an amino acid sequence as set forth in SEQ ID NO: 59.

In certain embodiments, the CAR comprises L6-Li and said L6-Li is located at a C terminal of the intracellular signaling domain. In certain embodiments, said L6-Li comprises an amino acid sequence as set forth in SEQ ID NO: 107.

In certain embodiments, the CAR comprises an amino acid sequence as set forth in any one of SEQ ID NOs: 64-71 and 74. In certain embodiments, the CAR comprises a nucleotide sequence as set forth in any one of SEQ ID NOs: 95-102 and 105.

In another aspect, this application provides an isolated nucleic acid molecule, said nucleic acid molecule comprising a nucleotide sequence encoding the CAR of this application.

In certain embodiments, said isolated nucleic acid molecule further comprises a promoter located at a 5′ terminal of the nucleotide sequence encoding said CAR. In certain embodiments, said promoter is a constitutive promoter. In certain embodiments, said promoter is an EF1α promoter.

In another aspect, this application provides a nucleic acid molecule encoding a CAR, said nucleic acid molecule encoding a CAR comprising a nucleotide sequence as set forth in any one of SEQ ID NOs: 95-102 and 105.

In another aspect, this application provides a vector comprising the nucleic acid molecule of this application.

In certain embodiments, the vector is selected from the group consisting of plasmids, retroviral vectors, and lentivirus vectors.

In another aspect, this application provides an immune effector cell comprising the CAR, the nucleic acid molecule, and/or the vector of this application. In certain embodiments, the immune effector cell is selected from T lymphocytes.

In another aspect, this application provides a method for preparing an immune effector cell, comprising: introducing the vector of this application into said immune effector cell.

In another aspect, this application provides a pharmaceutical composition comprising the CAR, the nucleic acid molecule, the vector, and/or the immune effector cell, and a pharmaceutically acceptable carrier.

In another aspect, this application further provides a use of the CAR, the nucleic acid molecule, the vector, the immune effector cell, and/or the pharmaceutical composition in the manufacture of a medicament, wherein said medicament is used for treating a disease and/or disorder associated with BCMA expression.

In certain embodiments, the disease or disorder associated with BCMA expression is a cancer or a malignant tumor. In certain embodiments, the disease or disorder associated with BCMA expression is a BCMA-positive tumor. In certain embodiments, the disease or disorder associated with BCMA expression is a solid tumor and/or a non-solid tumor. In certain embodiments, the disease or disorder associated with BCMA expression is myeloma. In certain embodiments, the disease or disorder associated with BCMA expression is multiple myeloma.

In another aspect, this application further provides a method for treating a disease or disorder associated with BCMA expression, comprising: administering the CAR, the nucleic acid molecule, the vector, the immune effector cell, and/or the pharmaceutical composition to a subject in need.

In certain embodiments, the disease or disorder associated with BCMA expression is a cancer or a malignant tumor. In certain embodiments, the disease or disorder associated with BCMA expression is a BCMA-positive tumor. In certain embodiments, the disease or disorder associated with BCMA expression is a solid tumor and/or a non-solid tumor. In certain embodiments, the disease or disorder associated with BCMA expression is myeloma. In certain embodiments, the disease or disorder associated with BCMA expression is multiple myeloma.

In another aspect, this application further provides the CAR, the nucleic acid molecule, the vector, the immune effector cell, and/or the pharmaceutical composition, used for treating a disease and/or disorder associated with BCMA expression.

In certain embodiments, the disease or disorder associated with BCMA expression is a cancer or a malignant tumor. In certain embodiments, the disease or disorder associated with BCMA expression is a BCMA-positive tumor. In certain embodiments, the disease or disorder associated with BCMA expression is a solid tumor and/or a non-solid tumor. In certain embodiments, the disease or disorder associated with BCMA expression is myeloma. In certain embodiments, the disease or disorder associated with BCMA expression is multiple myeloma.

Those skilled in the art can easily perceive other aspects and advantages of this application from the following detailed description. Only exemplary embodiments of this application are shown and described in detail below. As those skilled in the art will appreciate, from the content of the present application, those skilled in the art may make changes to the disclosed specific embodiments without departing from the spirit and scope of the invention to which the present application relates. Accordingly, the descriptions in the drawings and specification of this application are only exemplary and not restrictive.

### BRIEF DESCRIPTION OF THE DRAWINGS

The specific features of the invention referred to this application are shown in the appended claims. The features and advantages of the invention to which this application relates can be better understood with reference to the exemplary embodiments and the accompanying drawings described in detail below. The drawings are described briefly as follows.

FIG. 1 shows an embodiment of the composition of CAR elements in CAR lentivirus vectors and their connection sequences.

FIG. 2 shows the expansion fold of each group of BCMA CAR-T cell comprising the CAR of this application during the culture process.

FIG. 3 shows the proportion of CAR-positive cells in each group of BCMA CAR-T cell comprising the CAR of this application.

FIG. 4 shows the killing toxicity of each group of BCMA CAR-T cell comprising the CAR of this application against the target cell, where A shows the results of MM1s cell, and B shows the results of K562 cell.

FIG. 5 shows the IFN-γ cytokine secretion after co-incubation of each group of BCMA CAR-T cell comprising the CAR of this application with the MM1s cell.

FIG. 6 shows the targeted expansion ability of each group of BCMA CAR-T cell comprising the CAR of this application for the MM1s cell.

FIG. 7 shows the killing toxicity of BCMA CAR-T cell based on a L6-Li CAR structure against the MM1s cell.

FIG. 8 shows the IFN-γ cytokine secretion after co-incubation of each group of BCMA CAR-T cell based on the L6-Li CAR structure with the MM1s cell.

FIG. 9 shows the ability of BCMA CAR-T cells based on the L6-Li CAR structure to inhibit and decrease tumors in tumor-bearing mice, where A shows the results of the experimental group and B shows the results of the control group.

FIG. 10 shows the metabolism of BCMA CAR-T cells based on the L6-Li CAR structure to inhibit and decrease tumors in tumor-bearing mice, where A shows the results of the experimental group and B shows the results of the control group.

FIG. 11 shows IFN-γ cytokine secretion caused by BCMA CAR-T cells based on the L6-Li CAR structure in tumor-bearing mice, where A shows the results of the experimental group and B shows the results of the control group.

FIG. 12 shows IL-2 cytokine secretion caused by BCMA CAR-T cells based on the L6-Li CAR structure in tumor-bearing mice.

### DETAILED DESCRIPTION OF THE INVENTION

The implementation of the invention of this application will be described below with specific embodiments. Those skilled in the art can easily understand other advantages and effects of the invention of this application from the contents disclosed herein.

### Definition of Terms

As used herein, the term “BCMA” is used interchangeably with “CD269”, “BCM” and “TNFRSF17” and generally refers to B cell maturation antigen. The BCMA protein is a member of the tumor necrosis receptor family. As used herein, the term “BCMA” may include proteins containing mutations, for example, may include proteins containing point mutations, fragments, insertions, deletions and splice variants of full-length wild-type BCMA. The BCMA may include those from any vertebrate, for example, primates (e.g., humans or monkeys), rodents (mouse or rat), avian and/or livestock, and the like. As used herein, the term “BCMA” may also include a portion of the intact BCMA protein, as long as the relevant biological activity is retained. In this application, the BCMA may be human BCMA (GenBank ID: BAB60895.1). For example, human BCMA is typically a 184 amino acid long protein encoded by a 994 nucleotide long primary mRNA transcript (NM_001192.2).

As used herein, the term “binding domain” typically refers to a domain that (specifically) binds to a given target epitope or a given target site on a target molecule (antigen), or that interacts with, or recognizes a given target epitope or a given target site.

As used herein, the term “BCMA binding domain” typically refers to a domain that can specifically bind to a BCMA protein. For example, the BCMA binding domain may comprise a chimeric antigen receptor or a fragment thereof, an anti-BCMA antibody or an antigen-binding fragment thereof that specifically binds to a BCMA polypeptide expressed on a B cell. The terms “binding domain”, “extracellular domain”, “extracellular binding domain”, “antigen-specific binding domain” and “extracellular antigen-specific binding domain” are used interchangeably in this application and may comprise a domain or fragment of a CAR that has the ability to specifically bind to a target antigen of interest (e.g., BCMA). The BCMA binding domain can be of natural, synthetic, semi-synthetic or recombinant origin.

As used herein, the term “antibody” typically refers to an immunoglobulin or a fragment or derivative thereof and encompasses any polypeptide comprising an antigen-binding site, whether produced in vitro or in vivo. The term includes, but is not limited to, polyclonal, monoclonal, monospecific, multispecific, non-specific, humanized, single-stranded, chimeric, synthetic, recombinant, hybrid, mutated and transplanted antibodies. Unless otherwise modified by the term “intact”, as in “intact antibody”, the term “antibody” also includes antibody fragments, such as Fab, F(ab′)₂, Fv, scFv, Fd, dAb and other antibody fragments that maintain an antigen binding functionality, of, for example, specifically binding to BCMA. Typically, an antibody may comprise an immunoglobulin consisting of at least two heavy (H) chains and two light (L) chains interconnected by disulfide bonds, and comprise any molecule that contains an antigen-binding portion thereof. Each heavy chain consists of a heavy chain variable region (VH) and a heavy chain constant region. Each light chain consists of a light chain variable region (VL) and a light chain constant region. The VH and VL can be further distinguished into hypervariable regions called complementarity determining regions (CDRs), which are interspersed with more conserved regions called framework regions (FRs). Each VH and VL may be composed of three CDRs and four FR regions, which may be arranged in the following order from the amino terminal to the carboxyl terminal: FR1, CDR1, FR2, CDR2, FR3, CDR3 and FR4. The variable regions of the heavy and light chains contain binding domains that interact with an antigen.

As used herein, the term “scFv” typically refers to a single-chain antibody, which is an antibody composed of a heavy chain variable region and a light chain variable region that are linked directly or through a linking molecule (e.g., a linker peptide). The structure of the scFv, from the N terminal to the C terminal, may be heavy chain variable region-light chain variable region, light chain variable region-heavy chain variable region, heavy chain variable region-linker peptide-light chain variable region, or light chain variable region-linker peptide-heavy chain variable region.

As used herein, the term “chimeric antigen receptor (CAR)” typically refers to a fusion protein comprising an extracellular domain able to bind to an antigen and at least one intracellular domain. The CAR may be an antigen receptor formed by fusion of an antigen-binding region of an antibody that recognizes a tumor-associated antigen (TAA) and an intracellular signaling domain “immunoreceptor tyrosine-based activation motif (ITAM)”. The basic structure of a CAR includes a tumor-associated antigen (TAA) binding region (e.g., scFv), an extracellular hinge region, a transmembrane region, and an intracellular signaling domain. In this application, the CAR may be combined with an immune effector cell receptor activation intracellular domain based on the antigen (e.g., BCMA) specificity of the antibody. Immune effector cells genetically modified to express CAR can specifically recognize and eliminate malignant cells expressing target antigens.

As used herein, the term “signaling domain” generally refers to a domain located inside a cell that is capable of transducing a signal. The intracellular signaling domain described herein can transmit signals into cells. Typically, a signaling domain is any piece of contiguous amino acid sequence that directs a protein to find a target. For example, in certain embodiments, the intracellular signaling domain can be selected from the group consisting of CD3ζ intracellular domain, CD28 intracellular domain, CD28 intracellular domain, 4-1BB intracellular domain, and OX40 intracellular domain.

As used herein, the term “costimulation” generally refers to the source of lymphocyte activation secondary signals, usually produced by the interaction of costimulatory molecules and their receptors on the surface of immune cells (T cell/B cell or antigen presenting cell/T cell) involved in adaptive immunity. For example, complete activation of T cells depends on the action of dual signals and cytokines. The first signal of T cell activation comes from the specific binding of its receptor TCR to the antigen, that is, the T cell recognizes the antigen. The second signal of T cell activation comes from the costimulatory molecule, i.e., the interaction of a costimulatory molecule on APC with a corresponding receptor on the surface of the T cell.

As used herein, the term “costimulatory domain” typically refers to any amino acid sequence capable of interacting with a costimulatory molecule to produce costimulation.

As used herein, the term “hinge region” generally refers to a region in the CAR structure located between the antibody or antigen-binding fragment thereof (e.g., scFv) that specifically binds to BCMA and the transmembrane domain. The hinge region is typically derived from the IgG family, such as IgG1 and IgG4, and some are derived from IgD and CD8. Typically, the hinge region has a certain degree of flexibility, which affects the spatial constraint between the CAR molecule and its specific target, thereby affecting the contact between a CART cell and a tumor cell.

As used herein, the term “transmembrane domain” typically refers to a sequence in a cell surface protein that spans the cell membrane and may comprise a hydrophobic alpha helix. The transmembrane domain can be linked to the intracellular signaling domain to play a role in transmitting signals. The transmembrane domain typically contains three different structural regions: an N-terminal extracellular region, a middle conserved transmembrane stretching region, and a C-terminal cytoplasmic region. The transmembrane domain may also contain an intracellular or cytoplasmic region. The transmembrane domain described herein may be derived from any type I, type II or type III transmembrane protein.

As used herein, the term “signal peptide” typically refers to a leader sequence at the amino terminal (N-terminal) of the nascent CAR protein, which directs the nascent protein to the endoplasmic reticulum and subsequent surface expression during or after translation. The signal peptide is typically cleaved during this process. The signal peptide may be heterologous or homologous to the organism used to produce a polypeptide.

As used herein, the term “L6-Li” typically refers to the fusion protein of low-density lipoprotein receptor-related protein and leptin. For relevant information about L6-Li, please refer to Patent Application Publication No. WO2021057932A1. In this application, said L6-Li may comprise an amino acid sequence as set forth in SEQ ID NO: 107. In this application, the nucleic acid molecule encoding said L6-Li may comprise a nucleotide sequence as set forth in SEQ ID NO: 106.

As used herein, the term “promoter” generally refers to a deoxyribonucleic acid (DNA) sequence that enables the transcription of a specific gene. The promoter is recognized by RNA polymerase and begins transcription to synthesize RNA. In ribonucleic acid (RNA) synthesis, the promoter can interact with transcription factors that regulate gene transcription, controlling the initiation time and degree of gene expression (transcription). The promoter includes a core promoter region and a regulatory region, which are located in the regulatory sequence that controls gene expression and at upstream of the gene transcription start site (5′ direction of the DNA antisense strand). They have no compilation function themselves. According to their mode of action and function, they are divided into three categories: constitutive promoters (that remain continuously active in most or all tissues), specific (tissue specific or developmental period specific) promoters and inducible promoters (regulated by external chemical or physical signals).

As used herein, the term “specifically binding” generally refers to a measurable and reproducible interaction, such as the binding between a target and an antibody, that determines the presence of the target in the presence of a heterogeneous population of molecules, including biomolecules. For example, an antibody specifically binding to a target (or an epitope) may be an antibody that binds to that target with greater affinity, avidity, easier, and/or for a longer duration than it binds to other targets. In certain embodiments, the antibody specifically binds to an epitope on a protein that is conservative among proteins of different species. In another embodiment, the “specifically binding” includes, but does not require exclusive binding.

As used herein, the term “nucleic acid molecule” typically refers to an isolated form of nucleotides, deoxyribonucleotides or ribonucleotides of any length or their analogs. In certain embodiments, the nucleic acid molecule described herein may be isolated from the natural environment. In certain embodiments, the nucleic acid molecule described herein may be produced or synthesized by: (i) vitro amplification, for example, polymerase chain reaction (PCR) amplification; (ii) clonal recombination; (iii) purification, for example, enzymatic digestion and gel electrophoresis fractionation, or (iv) synthesis, for example, chemical synthesis. In certain embodiments, the isolated nucleic acid is a nucleic acid molecule prepared by recombinant DNA technology. In this application, nucleic acids encoding the antibodies or antigen-binding fragments thereof may be prepared by a variety of methods known in the art, including, but not limited to, overlap extension PCR using restriction fragment manipulation or synthetic oligonucleotides. For specific operations, please refer to Sambrook et al., Molecular Cloning, A Laboratory Manual, Cold Spring Harbor Laboratory Press, Cold Spring Harbor, N.Y., 1989; and Ausube et al., Current Protocols in Molecular Biology, Greene Publishing and Wiley-Interscience, New York N. Y., 1993.

As used herein, the terms “polypeptide” and “protein” are used interchangeably and generally refer to a polymer of amino acid residues. The term also applies to amino acid polymers in which one or more amino acid residues are analogs or mimetics of the corresponding naturally occurring amino acids, as well as naturally occurring amino acid polymers and homologues. The term may also include amino acid polymers modified, for example, by the addition of sugar residues to form glycoproteins or by phosphorylation. Polypeptides and proteins may be produced from naturally occurring and non-recombinant cells or from genetically engineered or recombinant cells, and may comprise molecules having the amino acid sequence of the native protein, or molecules having deletions, additions and/or substitutions of one or more amino acids of the native sequence. The terms “polypeptide” and “protein” particularly include sequences in which one or more amino acids of the antigen-binding proteins described herein are deleted, added and/or substituted.

When referring to the amino acid sequence of a protein or the nucleotide sequence of a nucleic acid molecule, the present application also includes homologs of these sequences. As used herein, the term “homologue” typically refers to an amino acid sequence or a nucleotide sequence that has certain homology to a wild-type amino acid sequence and a wild-type nucleotide sequence. The term “homology” may be equivalent to sequence “identity”. Homologous sequences may include amino acid sequences that may be at least 80% identical to the subject sequence 80%, 85%, 90%, 99.1%, 99.2%, 99.3%, 99.4%, 99.5%, 99.6%, 99.7%, 99.8%, or 99.9%. Typically, homologs will contain active sites, etc. that are identical to the subject amino acid sequence. Homology may be considered in terms of similarity (i.e., amino acid residues with similar chemical properties/functions), or homology may be expressed in terms of sequence identity. In this application, reference to a sequence having a percent identity over any one of the SEQ ID NOs of an amino acid sequence or a nucleotide sequence means a sequence having said percent identity over the entire length of the mentioned SEQ ID NO.

As used herein, the term “vector” refers to a nucleic acid delivery vehicle into which a polynucleotide encoding a protein can be inserted such that the protein can be expressed. The vector can be expressed by transforming, transducing or transfecting the host cell, so that the genetic material elements carried thereon can be expressed in the host cell. For example, the vector includes: plasmids; phagemids; cosmids; artificial chromosomes such as yeast artificial chromosomes (YACs), bacterial artificial chromosomes (BACs) or P1-derived artificial chromosomes (PACs); phages such as λ phage or M13 phage and animal viruses, etc. Animal virus species used as vectors include retroviruses (including lentiviruses), adenoviruses, adeno-associated viruses, herpesviruses (such as herpes simplex virus), poxviruses, baculoviruses, papillomaviruses, and papovavirus (e.g., SV40). A vector may comprise a variety of elements that control expression, including promoter sequence, transcriptional initiation sequence, enhancer sequence, selective element and reporter gene. In addition, the vector may also comprise replication initiation sites. The vector may also comprise components that assist its entry into cells, such as, but not exclusively, viral particles, liposomes, or protein coats.

As used herein, the term “immune effector cell” typically refers to cells involved in immune responses, for example, promoting immune effector responses. Examples of immune effector cells include T cells, e.g., α/β T cells and γ/δT T cells, B cells, natural killer (NK) cells, natural killer T (NKT) cells, mast cells, and bone marrow-derived phagocytes.

As used herein, the term “pharmaceutical composition” typically refers to a composition suitable for administration to patients, human patients. For example, the pharmaceutical composition of this application may include the immune effector cell, CAR, nucleic acid molecule or vector of this application, and optionally a pharmaceutically acceptable carrier. The pharmaceutical composition comprises a potent biologically active form of the active ingredient and does not comprise additional ingredients that would be unacceptably toxic to the subject to whom the formulation is to be administered.

As used herein, the term “pharmaceutically acceptable carrier” generally refers to one or more non-toxic materials that do not interfere with the effectiveness of the biological activity of the active ingredient. Such formulations may conventionally comprise salts, buffers, preservatives, compatible carriers, and optionally other therapeutic agents.

The term “disease or disorder associated with BCMA expression” includes, but is not limited to, diseases associated with BCMA expression, or disorders associated with BCMA-expressing cells, including tumor cells of various cancers. An individual with a disease or disorder associated with BCMA expression has abnormal BCMA expression (e.g., BCMA overexpression), or a cell has abnormal BCMA expression (e.g., BCMA overexpression) compared to a normal organism or a normal cell.

As used herein, the term “cancer or malignant tumor” typically refers to a neoplasm or solid lesion formed by abnormal cell growth. In this application, the tumor may be a solid tumor or a non-solid tumor (e.g., a hematological tumor).

As used herein, the term “BCMA-positive tumor” typically refers to tumors in which BCMA protein is overexpressed compared to normal cells. For example, the BCMA-positive tumor may include non-solid tumors. For example, the BCMA-positive tumor may include myeloma. For example, the BCMA-positive tumor may include multiple myeloma.

As used herein, the term “comprising” typically means including specifically specified features, but not excluding other elements.

As used herein, the term “about” generally refers to a variation within a range of 0.5%-10% above or below a specified value, for example, a variation within a range of 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5% or 10% above or below a specified value.

### DETAILED DESCRIPTION

In one aspect, this application provides a chimeric antigen receptor (CAR), comprising a BCMA binding domain, and said BCMA binding domain comprises an antibody that specifically binds to BCMA or an antigen-binding fragment of the antibody.

### BCMA Binding Domain

In this application, said antibody that specifically binds to BCMA or the antigen-binding fragment thereof in the CAR may comprise a heavy-chain complementarity determining region 3 (HCDR3), and said HCDR3 may comprise an amino acid sequence as set forth in SEQ ID NO: 25: DIRYVMDY (SEQ ID NO: 25). For example, said sequence can be divided according to Kabat.

In this application, said antibody or the antigen-binding fragment thereof may comprise a heavy-chain complementarity determining region 2 (HCDR2), and said HCDR2 may comprise an amino acid sequence as set forth in SEQ ID NO: 41: WINTETREPX₁₀YAYDFRX₁₇ (SEQ ID NO: 41), wherein X₁₀=A or T, X₁₇=G or S. For example, said sequence can be divided according to Kabat.

In this application, said HCDR2 may comprise an amino acid sequence as set forth in any one of SEQ ID NOs: 22-24.

In this application, said antibody or the antigen-binding fragment thereof may comprise a heavy-chain complementarity determining region 1 (HCDR1), and said HCDR1 may comprise an amino acid sequence as set forth in SEQ ID NO: 21: DYTIN (SEQ ID NO: 21). For example, said sequence can be divided according to Kabat.

In this application, said antibody or the antigen-binding fragment thereof may comprise an HCDR1, an HCDR2, and an HCDR3, and the amino acid sequences of said HCDR1, HCDR2 and HCDR3 may be as set forth in SEQ ID NO: 21, SEQ ID NO: 41, and SEQ ID NO: 25, respectively.

In this application, said antibody or the antigen-binding fragment thereof may comprise an HCDR1, an HCDR2, and an HCDR3, wherein said HCDR1 may comprise an amino acid sequence as set forth in SEQ ID NO: 21, said HCDR2 may comprise an amino acid sequence as set forth in any one of SEQ ID NOs: 22-24, and said HCDR3 may comprise an amino acid sequence as set forth in SEQ ID NO: 25.

In this application, said antibody or the antigen-binding fragment thereof may comprise an HCDR1, an HCDR2, and an HCDR3, wherein the amino acid sequences of said HCDR1, HCDR2, and HCDR3 may be as set forth in SEQ ID NO: 21, SEQ ID NO: 22, and SEQ ID NO: 25, respectively.

In this application, said antibody or the antigen-binding fragment thereof may comprise an HCDR1, an HCDR2, and an HCDR3, wherein the amino acid sequences of said HCDR1, HCDR2, and HCDR3 may be as set forth in SEQ ID NO: 21, SEQ ID NO: 23, and SEQ ID NO: 25, respectively.

In this application, said antibody or the antigen-binding fragment thereof may comprise an HCDR1, an HCDR2, and an HCDR3, wherein the amino acid sequences of said HCDR1, HCDR2, and HCDR3 may be as set forth in SEQ ID NO: 21, SEQ ID NO: 24, and SEQ ID NO: 25, respectively.

In this application, said antibody or the antigen-binding fragment that specifically binds to BCMA thereof in the CAR may comprise a light-chain complementarity determining region 3 (LCDR3), and said LCDR3 may comprise an amino acid sequence as set forth in SEQ ID NO: 40: LQSRX₅FPRX₉ (SEQ ID NO: 40), wherein X₅=I or R, X₉=H or T. For example, said sequence can be divided according to Kabat.

In this application, said LCDR3 may comprise an amino acid sequence as set forth in any one of SEQ ID NOs: 15-16.

In this application, said antibody or the antigen-binding fragment thereof may comprise a light-chain complementarity determining region 2 (LCDR2), and said LCDR2 may comprise an amino acid sequence as set forth in SEQ ID NO: 39: LGX₃NRX₆X₇ (SEQ ID NO: 39), where X₃=R or S, X₆=A or P, and X₇=A or S. For example, said sequence can be divided according to Kabat.

In this application, said LCDR2 may comprise an amino acid sequence as set forth in any one of SEQ ID NOs: 13-14.

In this application, said antibody or the antigen-binding fragment thereof may comprise a light-chain complementarity determining region 1 (LCDR1), and said LCDR1 may comprise an amino acid sequence as set forth in SEQ ID NO: 38: RASESVSVX₉GX₁₁X₁₂X₁₃X₁₄H (SEQ ID NO: 38), wherein X₉=I, P or S, X₁₁=A or I, X₁₂=H or S, X₁₃=F or L, and X₁₄=I or L. For example, said sequence can be divided according to Kabat.

In this application, said LCDR1 may comprise an amino acid sequence as set forth in any one of SEQ ID NOs: 10-12.

In this application, said antibody or the antigen-binding fragment thereof may comprise an LCDR1, an LCDR2, and an LCDR3, wherein the amino acid sequences of said LCDR1, LCDR2, and LCDR3 may be as set forth in SEQ ID NO: 38, SEQ ID NO: 39, and SEQ ID NO: 40, respectively.

In this application, said antibody or the antigen-binding fragment thereof may comprise an LCDR1, an LCDR2, and an LCDR3, wherein said LCDR1 may comprise an amino acid sequence as set forth in any one of SEQ ID NOs: 10-12, said LCDR2 may comprise an amino acid sequence as set forth in any one of SEQ ID NOs: 13-14, and said LCDR3 may comprise an amino acid sequence as set forth in any one of SEQ ID NOs: 15-16.

In this application, said antibody or the antigen-binding fragment thereof may comprise an LCDR1, an LCDR2, and an LCDR3, wherein the amino acid sequences of said LCDR1, LCDR2, and LCDR3 may be as set forth in SEQ ID NO: 10, SEQ ID NO: 13, and SEQ ID NO: 15, respectively.

In this application, said antibody or the antigen-binding fragment thereof may comprise an LCDR1, an LCDR2, and an LCDR3, wherein the amino acid sequences of said LCDR1, LCDR2, and LCDR3 may be as set forth in SEQ ID NO: 11, SEQ ID NO: 13, and SEQ ID NO: 16, respectively.

In this application, said antibody or the antigen-binding fragment thereof may comprise an LCDR1, an LCDR2, and an LCDR3, wherein the amino acid sequences of said LCDR1, LCDR2, and LCDR3 may be as set forth in SEQ ID NO: 12, SEQ ID NO: 13, and SEQ ID NO: 16, respectively.

In this application, said antibody or the antigen-binding fragment thereof may comprise an LCDR1, an LCDR2, and an LCDR3, wherein the amino acid sequences of said LCDR1, LCDR2, and LCDR3 may be as set forth in SEQ ID NO: 12, SEQ ID NO: 14, and SEQ ID NO: 16, respectively.

In this application, said antibody or the antigen-binding fragment thereof may comprise an HCDR1, an HCDR2, an HCDR3, an LCDR1, an LCDR2, and an LCDR3, wherein the amino acid sequences of said HCDR1, HCDR2, HCDR3, LCDR1, LCDR2, and LCDR3 may be as set forth in SEQ ID NO: 21, SEQ ID NO: 41, SEQ ID NO: 25, SEQ ID NO: 38, SEQ ID NO: 39, and SEQ ID NO: 40, respectively.

In this application, said antibody or the antigen-binding fragment thereof may comprise an HCDR1, an HCDR2, an HCDR3, an LCDR1, an LCDR2, and an LCDR3, wherein said HCDR1 may comprise an amino acid sequence as set forth in SEQ ID NO: 21, said HCDR2 may comprise an amino acid sequence as set forth in any one of SEQ ID NOs: 22-24, said HCDR3 may comprise an amino acid sequence as set forth in SEQ ID NO: 25, said LCDR1 may comprise an amino acid sequence as set forth in any one of SEQ ID NOs: 10-12, said LCDR2 may comprise an amino acid sequence as set forth in any one of SEQ ID NOs: 13-14, and said LCDR3 may comprise an amino acid sequence as set forth in any one of SEQ ID NOs: 15-16.

In this application, said antibody or the antigen-binding fragment thereof may comprise an HCDR1, an HCDR2, an HCDR3, an LCDR1, an LCDR2, and an LCDR3, wherein the amino acid sequences of said HCDR1, HCDR2, HCDR3, LCDR1, LCDR2, and LCDR3 may be as set forth in SEQ ID NO: 21, SEQ ID NO: 22, SEQ ID NO: 25, SEQ ID NO: 10, SEQ ID NO: 13, and SEQ ID NO: 15, respectively.

In this application, said antibody or the antigen-binding fragment thereof may comprise an HCDR1, an HCDR2, an HCDR3, an LCDR1, an LCDR2, and an LCDR3, wherein the amino acid sequences of said HCDR1, HCDR2, HCDR3, LCDR1, LCDR2, and LCDR3 may be as set forth in SEQ ID NO: 21, SEQ ID NO: 23, SEQ ID NO: 25, SEQ ID NO: 11, SEQ ID NO: 13, and SEQ ID NO: 16, respectively.

In this application, said antibody or the antigen-binding fragment thereof may comprise an HCDR1, an HCDR2, an HCDR3, an LCDR1, an LCDR2, and an LCDR3, wherein the amino acid sequences of said HCDR1, HCDR2, HCDR3, LCDR1, LCDR2, and LCDR3 may be as set forth in SEQ ID NO: 21, SEQ ID NO: 24, SEQ ID NO: 25, SEQ ID NO: 11, SEQ ID NO: 13, and SEQ ID NO: 16, respectively.

In this application, said antibody or the antigen-binding fragment thereof may comprise an HCDR1, an HCDR2, an HCDR3, an LCDR1, an LCDR2, and an LCDR3, wherein the amino acid sequences of said HCDR1, HCDR2, HCDR3, LCDR1, LCDR2, and LCDR3 may be as set forth in SEQ ID NO: 21, SEQ ID NO: 22, SEQ ID NO: 25, SEQ ID NO: 12, SEQ ID NO: 13, and SEQ ID NO: 16, respectively.

In this application, said antibody or the antigen-binding fragment thereof may comprise an HCDR1, an HCDR2, an HCDR3, an LCDR1, an LCDR2, and an LCDR3, wherein the amino acid sequences of said HCDR1, HCDR2, HCDR3, LCDR1, LCDR2, and LCDR3 may be as set forth in SEQ ID NO: 21, SEQ ID NO: 24, SEQ ID NO: 25, SEQ ID NO: 12, SEQ ID NO: 13, and SEQ ID NO: 16, respectively.

In this application, said antibody or the antigen-binding fragment thereof may comprise an HCDR1, an HCDR2, an HCDR3, an LCDR1, an LCDR2, and an LCDR3, wherein the amino acid sequences of said HCDR1, HCDR2, HCDR3, LCDR1, LCDR2, and LCDR3 may be as set forth in SEQ ID NO: 21, SEQ ID NO: 22, SEQ ID NO: 25, SEQ ID NO: 12, SEQ ID NO: 14, and SEQ ID NO: 16, respectively.

In this application, said antibody or the antigen-binding fragment thereof may comprise an HCDR1, an HCDR2, an HCDR3, an LCDR1, an LCDR2, and an LCDR3, wherein the amino acid sequences of said HCDR1, HCDR2, HCDR3, LCDR1, LCDR2, and LCDR3 may be as set forth in SEQ ID NO: 21, SEQ ID NO: 24, SEQ ID NO: 25, SEQ ID NO: 12, SEQ ID NO: 14, and SEQ ID NO: 16, respectively.

In this application, said antibody or the antigen-binding fragment thereof may comprise a frame region H-FR1, a C terminal of said H-FR1 is directly or indirectly linked to an N terminal of said HCDR1, and said H-FR1 may comprise an amino acid sequence as set forth in SEQ ID NO: 42: QVQLVQSGX₉EX₁₁KX₁₃PGASVKVSCKASGYX₂₈FA (SEQ ID NO: 42), wherein X₉=A or S, X₁₁=L or V, X₁₃=K or Q, and X₂₈=S or T. For example, said sequence can be divided according to Kabat.

In this application, said H-FR1 of said antibody or the antigen-binding fragment thereof may comprise an amino acid sequence as set forth in any one of SEQ ID NOs: 26-29.

In this application, said antibody or the antigen-binding fragment thereof may comprise a frame region H-FR2, and said H-FR2 is located between said HCDR1 and said HCDR2, and may comprise an amino acid sequence as set forth in SEQ ID NO: 43: WVRQAX₆GQGLEWX₁₃G (SEQ ID NO: 43), wherein X₆=P or T, and X₁₃=I or M. For example, said sequence can be divided according to Kabat.

In this application, said H-FR2 of said antibody or the antigen-binding fragment thereof may comprise an amino acid sequence as set forth in any one of SEQ ID NOs: 30-32.

In this application, said antibody or the antigen-binding fragment thereof may comprise a frame region H-FR3, and said H-FR3 is located between said HCDR2 and said HCDR3, and may comprise an amino acid sequence as set forth in SEQ ID NO: 44: RFX₃FX₅LX-TSX₁₀STAYLX₁₆X₁₇SSLX₂₁X₂₂EDTAVYYCAR (SEQ ID NO: 44), wherein X₃=T or V, X₅=S or T, X₇=D or N, X₁₀=A, I or V, X₁₆=E or Q, X₁₇=I or L, X₂₁=K or R, and X₂₂=A or S. For example, said sequence can be divided according to Kabat.

In this application, said H-FR3 of said antibody or the antigen-binding fragment thereof may comprise an amino acid sequence as set forth in any one of SEQ ID NOs: 33-36.

In this application, said antibody or the antigen-binding fragment thereof may comprise a frame region H-FR4, an N terminal of said H-FR4 is linked to a C terminal of said HCDR3, and said H-FR4 may comprise an amino acid sequence as set forth in SEQ ID NO: 37: WGQGTLVTVSS (SEQ ID NO: 37). For example, said sequence can be divided according to Kabat.

In this application, said antibody or the antigen-binding fragment thereof may comprise an H-FR1, an H-FR2, an H-FR3, and an H-FR4, wherein the amino acid sequences of said H-FR1, H-FR2, H-FR3, and H-FR4 may be as set forth in SEQ ID NO: 42, SEQ ID NO: 43, SEQ ID NO: 44, and SEQ ID NO: 37, respectively.

In this application, said antibody or the antigen-binding fragment thereof may comprise an H-FR1, an H-FR2, an H-FR3, and an H-FR4, wherein said H-FR1 may comprise an amino acid sequence as set forth in any one of SEQ ID NOs: 26-29, said H-FR2 may comprise an amino acid sequence as set forth in any one of SEQ ID NOs: 30-32, said H-FR3 may comprise an amino acid sequence as set forth in any one of SEQ ID NOs: 33-36, and said H-FR4 may comprise an amino acid sequence as set forth in SEQ ID NO: 37.

In this application, said antibody or the antigen-binding fragment thereof may comprise any set of H-FR1, H-FR2, H-FR3, and H-FR4, selected from the groups consisting of: (1) H-FR1: SEQ ID NO: 26, H-FR2: SEQ ID NO: 30, H-FR3: SEQ ID NO: 33, and H-FR4: SEQ ID NO: 37; (2) H-FR1: SEQ ID NO: 27, H-FR2: SEQ ID NO: 31, H-FR3: SEQ ID NO: 34, and H-FR4: SEQ ID NO: 37; (3) H-FR1: SEQ ID NO: 28, H-FR2: SEQ ID NO: 32, H-FR3: SEQ ID NO: 35, and H-FR4: SEQ ID NO: 37; and (4) H-FR1: SEQ ID NO: 29, H-FR2: SEQ ID NO: 30, H-FR3: SEQ ID NO: 36, and H-FR4: SEQ ID NO: 37.

In this application, said antibody or the antigen-binding fragment thereof may comprise an antibody weight chain variable region (VH), and said VH may comprise an amino acid sequence as set NO: forth in SEQ ID 46: QVQLVQSGX₉EX₁₁KX₁₃PGASVKVSCKASGYX₂₈FADYTINWVRQAX₄₁GQGLEWX₄₈GWINTETREPX₅₉YAYDFRX₆₆RFX₆₉FX₇₁LX₇₃TSX₇₆STAYLX₈₂X₈₃SSLX₈₇X₈₈EDTAV YYCARDIRYVMDYWGQGTLVTVSS (SEQ ID NO: 46), wherein X₉=A or S, X₁₁=L or V, X₁₃=K or Q, X₂₈=S or T, X₄₁=P or T, X₄₈=I or M, X₅₉=A or T, X₆₆=G or S, X₆₉=T or V, X₇₁=S or T, X₇₃=D or N, X₇₆=A, I or V, X₈₂=E or Q, X₈₃=I or L, X₈₇=K or R, and X₈₈=A or S. For example, said sequence can be divided according to Kabat.

In this application, said VH of said antibody or the antigen-binding fragment thereof may comprise an amino acid sequence as set forth in any one of SEQ ID NOs: 5-9.

In this application, said antibody or the antigen-binding fragment thereof may comprise a frame region L-FR1, a C terminal of said L-FR1 is directly or indirectly linked to an N terminal of said LCDR1, and said L-FR1 may comprise an amino acid sequence as set forth in SEQ ID NO: 17: EIVLTQSPATLSLSPGERATLSC (SEQ ID NO: 17). For example, said sequence can be divided according to Kabat.

In this application, said antibody or the antigen-binding fragment thereof may comprise a frame region L-FR2, said L-FR2 is located between said LCDR1 and said LCDR2, and said L-FR2 may comprise an amino acid sequence as set forth in SEQ ID NO: 18: WYQQKPGQAPRLLIY (SEQ ID NO: 18). For example, said sequence can be divided according to Kabat.

In this application, said antibody or the antigen-binding fragment thereof may comprise a frame region L-FR3, and said L-FR3 is located between said LCDR2 and said LCDR3, and may comprise an amino acid sequence as set forth in SEQ ID NO: 19: GIPARFSGSGSGTDFTLTISSLEPEDAAIYYC (SEQ ID NO: 19). For example, said sequence can be divided according to Kabat.

In this application, said antibody or the antigen-binding fragment thereof may comprise a frame region L-FR4, an N terminal of said L-FR4 is linked to a C terminal of said LCDR3, and said L-FR4 may comprise an amino acid sequence as set forth in SEQ ID NO: 20: FGQGTKLEIK (SEQ ID NO: 20). For example, said sequence can be divided according to Kabat.

In this application, said antibody or the antigen-binding fragment thereof may comprise an L-FR1, an L-FR2, an L-FR3, and an L-FR4, wherein the amino acid sequences of said L-FR1, L-FR2, L-FR3, and L-FR4 may be as set forth in SEQ ID NO: 17, SEQ ID NO: 18, SEQ ID NO: 19, and SEQ ID NO: 20, respectively.

In this application, said antibody or the antigen-binding fragment thereof may comprise an antibody light chain variable region (VL), and said VL may comprise an amino acid sequence as set forth in SEQ ID NO: 45: EIVLTQSPATLSLSPGERATLSCRASESVSVX₃₂GX₃₄X₃₅X₃₆X₃₇HWYQQKPGQAPRLL IYLGX₅₆NRX₅₉X₆₀GIPARFSGSGSGTDFTLTISSLEPEDAAIYYCLQSRX₉₇FPRX₁₀₁FG QGTKLEIK (SEQ ID NO: 45), wherein X₃₂=I, P or S, X₃₄=A or I, X₃₅=H or S, X₃₆=F or L, X₃₇=I or L, X₅₆=R or S, X₅₉=A or P, X₆₀=A or S, X₉₇=I or R, and X₁₀₁=H or T. For example, said sequence can be divided according to Kabat.

In this application, said VL of said antibody or the antigen-binding fragment thereof may comprise an amino acid sequence as set forth in any one of SEQ ID NOs: 1-4.

In this application, said antibody or the antigen-binding fragment thereof may comprise a VH and a VL, said VH may comprise an amino acid sequence as set forth in SEQ ID NO: 46, and said VL may comprise an amino acid sequence as set forth in SEQ ID NO: 45.

In this application, said antibody or the antigen-binding fragment thereof may comprise a VH and a VL, said VH may comprise an amino acid sequence as set forth in any one of SEQ ID NOs: 5-9, and said VL may comprise an amino acid sequence as set forth in any one of SEQ ID NOs: 1-4.

In this application, said antibody or the antigen-binding fragment thereof may comprise a VH and a VL, said VH may comprise an amino acid sequence as set forth in SEQ ID NO: 5, and said VL may comprise an amino acid sequence as set forth in SEQ ID NO: 1.

In this application, said antibody or the antigen-binding fragment thereof may comprise a VH and a VL, said VH may comprise an amino acid sequence as set forth in SEQ ID NO: 6, and said VL may comprise an amino acid sequence as set forth in SEQ ID NO: 2.

In this application, said antibody or the antigen-binding fragment thereof may comprise a VH and a VL, said VH may comprise an amino acid sequence as set forth in SEQ ID NO: 7, and said VL may comprise an amino acid sequence as set forth in SEQ ID NO: 2.

In this application, said antibody or the antigen-binding fragment thereof may comprise a VH and a VL, said VH may comprise an amino acid sequence as set forth in SEQ ID NO: 8, and said VL may comprise an amino acid sequence as set forth in SEQ ID NO: 2.

In this application, said antibody or the antigen-binding fragment thereof may comprise a VH and a VL, said VH may comprise an amino acid sequence as set forth in SEQ ID NO: 9, and said VL may comprise an amino acid sequence as set forth in SEQ ID NO: 3.

In this application, said antibody or the antigen-binding fragment thereof may comprise a VH and a VL, said VH may comprise an amino acid sequence as set forth in SEQ ID NO: 8, and said VL may comprise an amino acid sequence as set forth in SEQ ID NO: 3.

In this application, said antibody or the antigen-binding fragment thereof may comprise a VH and a VL, said VH may comprise an amino acid sequence as set forth in SEQ ID NO: 9, and said VL may comprise an amino acid sequence as set forth in SEQ ID NO: 4.

In this application, said antibody or the antigen-binding fragment thereof may comprise a VH and a VL, said VH may comprise an amino acid sequence as set forth in SEQ ID NO: 8, and said VL may comprise an amino acid sequence as set forth in SEQ ID NO: 4.

In this application, said antibody or the antigen-binding fragment thereof may comprise a scFv, and said scFv may comprise said VH and said VL. In said scFv, the C terminal of said VH and the N terminal of said VL may be linked directly, for example, in-frame. In said scFv, the C terminal of said VH and the N terminal of said VL may be linked indirectly, for example, by a linker (e.g., a linker peptide). In said scFv, the C terminal of said VL and the N terminal of said VH may be linked directly, for example, in-frame. In said scFv, the C terminal of said VL and the N terminal of said VH may be linked indirectly, for example, by a linker (e.g., a linker peptide).

In this application, the BCMA domain of the CAR may comprise a VH, a VL and a linker peptide, said VH may comprise an amino acid sequence as set forth in SEQ ID NO: 5, said VL may comprise an amino acid sequence as set forth in SEQ ID NO: 1, and said linker peptide may comprise an amino acid sequence as set forth in SEQ ID NO: 58.

In this application, the BCMA domain of the CAR may comprise a VH, a VL and a linker peptide, said VH may comprise an amino acid sequence as set forth in SEQ ID NO: 6, said VL may comprise an amino acid sequence as set forth in SEQ ID NO: 2, and said linker peptide may comprise an amino acid sequence as set forth in SEQ ID NO: 58.

In this application, the BCMA domain of the CAR may comprise a VH, a VL and a linker peptide, said VH may comprise an amino acid sequence as set forth in SEQ ID NO: 7, said VL may comprise an amino acid sequence as set forth in SEQ ID NO: 2, and said linker peptide may comprise an amino acid sequence as set forth in SEQ ID NO: 58.

In this application, the BCMA domain of the CAR may comprise a VH, a VL and a linker peptide, said VH may comprise an amino acid sequence as set forth in SEQ ID NO: 8, said VL may comprise an amino acid sequence as set forth in SEQ ID NO: 2, and said linker peptide may comprise an amino acid sequence as set forth in SEQ ID NO: 58.

In this application, the BCMA domain of the CAR may comprise a VH, a VL and a linker peptide, said VH may comprise an amino acid sequence as set forth in SEQ ID NO: 9, said VL may comprise an amino acid sequence as set forth in SEQ ID NO: 3, and said linker peptide may comprise an amino acid sequence as set forth in SEQ ID NO: 58.

In this application, the BCMA domain of the CAR may comprise a VH, a VL and a linker peptide, said VH may comprise an amino acid sequence as set forth in SEQ ID NO: 8, said VL may comprise an amino acid sequence as set forth in SEQ ID NO: 3, and said linker peptide may comprise an amino acid sequence as set forth in SEQ ID NO: 58.

In this application, the BCMA domain of the CAR may comprise a VH, a VL and a linker peptide, said VH may comprise an amino acid sequence as set forth in SEQ ID NO: 9, said VL may comprise an amino acid sequence as set forth in SEQ ID NO: 4, and said linker peptide may comprise an amino acid sequence as set forth in SEQ ID NO: 58.

In this application, the BCMA domain of the CAR may comprise a VH, a VL and a linker peptide, said VH may comprise an amino acid sequence as set forth in SEQ ID NO: 8, said VL may comprise an amino acid sequence as set forth in SEQ ID NO: 4, and said linker peptide may comprise an amino acid sequence as set forth in SEQ ID NO: 58.

In this application, the BCMA domain of the CAR may comprise a VH, a VL and a linker peptide, the C terminal of said VL may be linked to the N terminal of the VH by a linker peptide, and the linker peptide may comprise an amino acid sequence as set forth in SEQ ID NO: 58.

In this application, the BCMA domain of the CAR may comprise a scFv, and said scFv may comprise an amino acid sequence as set forth in any one of SEQ ID Nos: 39-49. In this application, said scFv may comprise an amino acid sequence at least 80% (e.g., at least 85%, 90%, 91%, 92%, 93%, 94%, 95%, 96%, 97%, 98%, 99% or higher) identical to the amino acid sequence as set forth in any one of SEQ ID NOs: 39-49.

### Chimeric Antigen Receptor (CAR)

In this application, the CAR may further comprise an intracellular domain in addition to the extracellular BCMA domain.

In some cases, the CAR may comprise an intracellular costimulatory signaling domain that can provide a stimulatory signal. Said costimulatory signaling domain may include, but be not limited to, costimulatory molecules composed of costimulatory signaling regions in the following group consisting of CD137, CD28, 4-1BB, OX-40, and ICOS, and combinations thereof. For example, said costimulatory domain may be a CD137-derived stimulatory domain. For example, said costimulatory domain may comprise an amino acid sequence as set forth in SEQ ID NO: 62.

In some cases, the CAR may comprise an intracellular signaling domain, which may comprise a domain containing at least one ITAM. Said intracellular signaling domain can transmit an activation signal into the cell. An exemplary signaling domain may be signaling domains derived from proteins elected from the group consisting of, but not limited to, CD35, CD38, CD3γ, CD3ε, CD79a, CD79b, FcεRIγ, FcεRIβ, FcγRIIa, bovine leukemia virus gp30 activation region, Epstein-Barr virus (EBV) LMP2A, simian immunodeficiency virus PBj14Nef, Kaposi's sarcoma-associated herpesvirus (HSKV), DAP10 and DAP-12, and variants thereof. For example, said intracellular signaling domain may be a CD3ζ-derived signaling domain. For example, said intracellular signaling domain may comprise an amino acid sequence as set forth in SEQ ID NO: 63.

In some cases, the CAR may comprise a transmembrane domain which is a sequence in a cell surface protein that spans the cell membrane. The transmembrane domain may comprise a hydrophobic alpha helix. The transmembrane domain may be derived from CD28 and has good stability. The transmembrane domain may be derived from any type I transmembrane protein. The ansmembrane domain may be a synthetic sequence predicted to form a hydrophobic helix. Said transmembrane domain may comprise transmembrane domains derived from one or more proteins selected from the group consisting of: CD28, CD3e, CD45, CD4, CD5, CD8, CD9, CD16, CD22, CD33, CD37, CD64, CD80, CD86, CD134, CD137, and CD154. For example, said transmembrane domain may be a CD8-derived transmembrane domain. For example, said transmembrane domain may comprise an amino acid sequence as set forth in SEQ ID NO: 61.

In some cases, the CAR may comprise a hinge region, which may be located between said extracellular targeting moiety and said transmembrane domain. Said hinge region may comprise a hinge region derived from one or more proteins selected from the group consisting of: CD28, IgG1, IgG4, IgD, 4-1BB, CD4, CD27, CD7, CD8 alpha, PD-1, ICOS, OX40, NKG2D, NKG2C, FcεRIγ, BTLA, GITR, DAP10, CD40L, TIM1, CD226, SLAM, CD30, and LIGHT. For example, said hinge region may be derived from CD8. For example, said hinge region may comprise an amino acid sequence as set forth in SEQ ID NO: 60.

In this application, the CAR may further comprise a signal peptide, for example, human CD8-derived signal peptide, at the N terminal of the BCMA domain. For example, the signal peptide may comprise an amino acid sequence as set forth in SEQ ID NO: 59.

In this application, from the N terminal to the C terminal, the CAR may sequentially comprise a BCMA targeting moiety (e.g., said antigen-binding protein, e.g., said scFv), said hinge region, said transmembrane domain, said costimulatory signaling domain, and said signaling domain.

In this application, from the N terminal to the C terminal, the CAR may sequentially comprise said scFv, said CD8-derived hinge region, said CD8-derived transmembrane domain, said CD137-derived costimulatory signaling domain and said CD3ζ-derived signaling domain. For example, the CAR may comprise an amino acid sequence as set forth in any one of SEQ ID NOs: 64-71.

In this application, the C terminal of the CAR may be linked to a leptin and/or a functional fragment thereof, and/or low-density lipoprotein receptor-related protein or a fragment thereof. For example, said leptin may comprise secretory leptins. For example, low-density lipoprotein receptor-related protein 6.

In this application, said CAR, said leptin and/or the functional fragment thereof, and/or said low-density lipoprotein receptor-related protein or the fragment thereof may be linked by a self-cleaving peptide (e.g., a 2A peptide such as T2A, P2A, and E2A).

In this application, the C terminal of the CAR may be linked to L6-Li. For example, said L6-Li may comprise an amino acid sequence as set forth in SEQ ID NO: 107. For example, the nucleic acid molecule encoding said L6-Li may comprise a nucleotide sequence as set forth in SEQ ID NO: 106.

In this application, from the N terminal to the C terminal, the CAR may sequentially comprise a BCMA targeting moiety (e.g., said antigen-binding protein, e.g., said scFv), said hinge region, said transmembrane domain, said costimulatory signaling domain, said signaling domain, and said L6-Li.

For example, from the N terminal to the C terminal, the CAR may sequentially comprise said scFv, said CD8-derived hinge region, said CD8-derived transmembrane domain, said CD137-derived costimulatory signaling domain, said CD3 ζ-derived signaling domain, and said L6-Li. For example, the CAR may comprise an amino acid sequence as set forth in SEQ ID NO: 74.

### Nucleic Acid Molecules, Vectors, Cells, Preparation Methods and Pharmaceutical Compositions

In another aspect, this application further provides one or more isolated nucleic acid molecules that may encode the chimeric antigen receptor (CAR) of this application. The one or more isolated nucleic acid molecules of this application may be an isolated form of nucleotides, deoxyribonucleotides or ribonucleotides of any length, or analogs isolated from their natural environment or artificially synthesized, but can encode the CAR of this application. For example, the nucleic acid molecule may comprise a nucleotide sequence as set forth in any one of SEQ ID NOs: 95-102 and 105.

In another aspect, this application further provides a vector, which may comprise the nucleic acid molecule described herein. The vector can be expressed by transforming, transducing or transfecting a host cell so that the genetic material elements carried thereon can be expressed in the host cell. For example, the vector may include: plasmids; phagemids; cosmids; artificial chromosomes such as yeast artificial chromosomes (YACs), bacterial artificial chromosomes (BACs) or P1-derived artificial chromosomes (PACs); phages such as λ phage or M13 phage and animal viruses, etc. Animal virus species used as vectors include retroviruses (including lentiviruses), adenoviruses, adeno-associated viruses, herpesviruses (such as herpes simplex virus), poxviruses, baculoviruses, papillomaviruses, and papovavirus (e.g., SV40). For another example, said vector may comprise a variety of elements that control expression, including promoter sequence, transcriptional initiation sequence, enhancer sequence, selective element and reporter gene. In addition, said vector may also comprise replication initiation sites. In addition, said vector may also comprise components that assist its entry into cells, such as, but not exclusively, viral particles, liposomes, or protein coats.

In another aspect, this application further provides an immune effector cell that may comprise the CAR, the nucleic acid molecule, and/or the vector of this application. Said cell may include a progeny of an individual cell. Due to natural, accidental or deliberate mutations, the progeny cell may not be necessarily identical (either in the morphology of the total DNA complement or in the genome) to the original parent cell. In certain embodiments, said cell may further include cells transfected in vitro with vector of this application. In certain embodiments, said cell may be a mammalian cell. In certain embodiments, said immune effector cell includes T lymphocytes, for example, α/β T lymphocytes and γ/δ T lymphocytes. In certain embodiments, said immune effector cell may include natural killer (NK) cells, natural killer T (NKT) cells, mast cells and bone marrow-derived phagocytes.

In one aspect, this application provides a modified immune effector cell, comprising a chimeric antigen receptor and/or a coding element thereof, or comprising a T cell receptor and/or a coding element thereof, and the modified immune cell further comprises: a leptin and/or a functional fragment thereof; and/or a leptin receptor and/or a functional fragment thereof. Compared with immune cells without corresponding modification, the modified immune cell has increased expression of said leptin receptor and/or the functional fragment thereof. In this application, the immune effector cell may comprise leptin and/or a functional fragment thereof, and/or a leptin receptor and/or a functional fragment thereof, and a low-density lipoprotein receptor-related protein or a fragment thereof. For the modification of immune effector cells, please refer to WO2021057932A1. The description in WO2021057932A1 can also be applied to this application.

In another aspect, this application further provides a method for preparing the immune effector cell described herein. The method may include: introducing the isolated nucleic acid molecule of this disclosure or the vector of this application into the immune effector cell.

In another aspect, this application further provides a composition, which may comprise the immune effector cell described herein. In certain embodiments, the composition may further comprise optionally a pharmaceutically acceptable carrier. In certain embodiments, the acceptable ingredients of the composition are not toxic to recipients at the doses and concentrations used. The pharmaceutical composition of this application includes, but is not limited to, liquid, frozen and lyophilized compositions. In certain embodiments, said pharmaceutically acceptable adjuvant may include any and all solvents, dispersion media, isotonic agents, and absorption delaying agents that are compatible with said immune effector cell, are generally safe and nontoxic, and are neither biologically nor otherwise undesirable.

In certain embodiments, the composition may be administered by routes including parenteral, transdermal, intracavity, intraarterial, intrathecal and/or intranasal administration or may be directly injected into tissues. For example, the composition may be administered to a patient or subject by infusion or injection. In certain embodiments, the pharmaceutical composition may be administered by different means, such as intravenous, intraperitoneal, subcutaneous, intramuscular, topical or intradermal administration.

### Treatment Method

In another aspect, this application further provides a use of the chimeric antigen receptor of this application, the nucleic acid molecule of this application, the vector of this application, the immune effector cell of this application and/or the composition of this application in the manufacture of a medicament, wherein said medicament is used for treating a disease or disorder associated with BCMA expression.

In this application, the disease or disorder associated with BCMA expression is a cancer or a malignant tumor. For example, the disease or disorder associated with BCMA expression may include BCMA-positive tumors. For example, said cancer or malignant tumor may include solid tumors or non-solid tumors. For example, said cancer or malignant tumor may be a non-solid tumor. For example, said cancer or malignant tumor may include myeloma. for example, said myeloma may be multiple myeloma.

In another aspect, this application further provides a method for preventing, alleviating or treating a tumor. The method may comprise: administering the CAR, the nucleic acid molecule, the vector, the immune effector cell and/or the pharmaceutical composition of this application to a subject in need. In this application, the administration may be carried out by different means, such as intravenous, intratumoral, intraperitoneal, subcutaneous, intramuscular, topical or intradermal administration.

In this application, said subject may include humans and non-human animals. For example, said subject may include, but is not limited to, cats, dogs, horses, pigs, cows, sheep, rabbits, mice, rats or monkeys. Without wishing to be bound by any theory, the following examples are only for illustrating various technical solutions of the invention of this application, but are not intended to limit the scope of the invention of this application.

### EXAMPLES

### Example 1 Production of scFv

Recombinant human BCMA-hFc and BCMA-His proteins, prepared by Oricell Therapeutics Co., Ltd., were alternately sorted with the natural human phage antibody library for a total of four rounds, where the human BCMA-hFc represents a protein formed by linking human BCMA to human Fc, the amino acid sequence of human BCMA protein (GenBank ID: BAB60895.1) can be found in NCBI's Official website, and the amino acid sequence of human Fc is as set forth in SEQ ID NO: 57. After four rounds of sorting, significant enrichment was observed. Phage antibody clones obtained by sorting were identified using ELISA. The obtained positive antibody sequence was then subjected to affinity testing and sequencing identification. The sorted candidate antibodies were subjected to one to two rounds of affinity evolution and also subjected to affinity testing and binding activity identification by flow cytometry again, and a total of eight phage antibody clones that can specifically bind to human BCMA were obtained and named hBCMA-01, hBCMA-02, hBCMA-15, hBCMA-17, hBCMA-19, hBCMA-20, hBCMA-22, and hBCMA-23, respectively. After sequencing, the nucleic acid sequences of their VL and VH were obtained. The nucleic acid sequences of the VL and the VH were linked by a linker peptide nucleic acid sequence having a nucleic acid sequence as set forth in SEQ ID NO: 58 and an amino acid sequence as set forth in SEQ ID NO: 85 to form a single-chain antibody (scFv). The scFv sequence of the positive control was huBCMA-10 disclosed in Patent Application US 2017/0226216 A1. The nucleic acid sequences of all the above nine scFvs (including huBCMA-10) were delivered to GenScript, Nanjing for gene synthesis, and a forward primer having a nucleic acid sequence as set forth in SEQ ID NO: 87 was added to the 5′ terminal of the scFv nucleic acid sequence and a reverse primer having a nucleic acid sequence as set forth in SEQ ID NO: 88 was added to the 3′ terminal of the scFv nucleic acid sequence for molecular construction in the next step of homologous recombination. The VH and VL gene sequences of these eight antibody clones and the positive control are shown in Table 1.

TABLE 1
VH and VL sequences of eight antibody
clones and the positive control

| | Nucleic acid sequence | | Amino acid sequence | |
|---|---|---|---|---|
| scFv | (SEQ ID NO:) | | (SEQ ID NO:) | |
| sequence | VL | VH | VL | VH |
| hBCMA-01 | 75 | 80 | 1 | 5 |
| hBCMA-02 | 76 | 81 | 2 | 6 |
| hBCMA-15 | 76 | 82 | 2 | 7 |
| hBCMA-17 | 76 | 83 | 2 | 8 |
| hBCMA-19 | 77 | 84 | 3 | 9 |
| hBCMA-20 | 77 | 83 | 3 | 8 |
| hBCMA-22 | 78 | 84 | 4 | 9 |
| hBCMA-23 | 78 | 83 | 4 | 8 |
| huBCMA-10 | 79 | 85 | 47 | 48 |

### Example 2 Construction of CAR Lentivirus Vectors

The CAR lentivirus core empty plasmid (hereinafter referred to as CAR lentivirus empty vector, constructed by Oricell Therapeutics Co., Ltd., or CAR lentivirus vector (comprising the L6-Li element, please refer to Patent Application Publication No. WO2021057932A1; the amino acid sequence of L6-Li is as set forth in SEQ ID NO: 107) (hereinafter referred to as CAR-L6-Li lentivirus vector, constructed by OriCell Therapeutics, Shanghai) was double-digested by using SphI and NotI endonuclease (purchased from NEB). The former produced a 7749 bp linearized fragment, and the latter produced a 9605 bp linearized fragment.

After gel cutting and recovery, the produced 7749 bp linearized fragment and the nucleic acid sequence fragment of the scFv (including homology arms at both ends) obtained in Example 1 were mixed in a molar ratio of 1:3 (not more than 10 μl in volume), and after homologous recombination, E. coli DH5α competent cell was transformed. Single colonies were picked, shaken, plasmid extracted and sent for sequencing to verify whether the inserted scFv sequence was correct.

After gel cutting and recovery, the produced 9605 bp linearized fragment and the nucleic acid sequence fragments of the scFvs in hBCMA-22 and huBCMA-10 (including homology arms at both ends) obtained in Example 1 were mixed in a molar ratio of 1:3 (not more than 10 μl in volume), and after homologous recombination, E. coli DH5α competent cell was transformed. Single colonies were picked, shaken, plasmid extracted and sent for sequencing identification.

The CAR and various elements of CAR-L6-Li in the CAR lentivirus empty vector and h (u) BCMA-XX core plasmid (hereinafter referred to as h (u) BCMA-XX core plasmid) and connection sequences are shown in FIG. 1. The amino acid sequences and nucleic acid sequences of the various elements are shown in Table 2. The CAR portion of the CAR lentivirus empty vector comprises the signal peptide-CD8-derived hinge region-CD8-derived transmembrane domain-CD137-derived costimulatory domain-CD3 ζ-derived intracellular signaling domain in sequence. Based on the CAR lentivirus empty vector, the CAR vector has a scFv targeting BCMA inserted between the signal peptide and the CD8-derived hinge region. Based on the CAR vector, the CAR-L6-Li vector has a L6-Li sequence linked to the 3′ terminal of CD3ζ. The 5′ terminal of each vector is linked to the EF1α promoter. The sequences of the CAR portions in the plasmid vectors are shown in Table 3.

TABLE 2
Sequences of various elements in CAR and CAR-L6-Li

| | Nucleotide | Amino acid |
|---|---|---|
| | sequence | sequence |
| Element | (SEQ ID NO) | (SEQ ID NO) |
| Signal peptide | 90 | 59 |
| CD8-derived hinge region | 91 | 60 |
| CD8-derived | 92 | 61 |
| transmembrane domain | | |
| CD137-derived | 93 | 62 |
| costimulatory domain | | |
| CD3ζ-derived intracellular | 94 | 63 |
| signaling domain | | |
| L6-Li | SEQ ID NO: 106 | SEQ ID NO: 107 |

TABLE 3
Sequences of CAR portions in plasmid vectors

| | Nucleotide sequence | Amino acid sequence |
|---|---|---|
| | (SEQ ID NO) | (SEQ ID NO) |
| hBCMA-01 CAR | 95 | 64 |
| hBCMA-02 CAR | 96 | 65 |
| hBCMA-15 CAR | 97 | 66 |
| hBCMA-17 CAR | 98 | 67 |
| hBCMA-19 CAR | 99 | 68 |
| hBCMA-20 CAR | 100 | 69 |
| hBCMA-22 CAR | 101 | 70 |
| hBCMA-23 CAR | 102 | 71 |
| huBCMA-10 CAR | 103 | 72 |
| huBCMA-10-L6-Li CAR | 104 | 73 |
| huBCMA-22-L6-Li CAR | 105 | 74 |

### Example 3 Lentivirus Packaging

As an example, the system used to construct the lentivirus vector of the present invention was the third generation. The system was composed of three plasmids, i.e., a packaging plasmid psPAX2 (Addgene, plasmid number #12260) encoding Gag-Pol protein and Rev protein, a PMD2.G plasmid (Addgene, plasmid number #12259) encoding an evelope protein VSV-G (Addgene, plasmid number #12259), and a core plasmid, i.e., the CAR lentivirus plasmids containing the scFv sequence in the above Examples 1-2 (i.e., hBCMA-01, hBCMA-02, hBCMA-15, hBCMA-17, hBCMA-19, hBCMA-20, hBCMA-22, hBCMA-23, and huBCMA-10). The expression of the CAR gene in each CAR lentivirus plasmid was controlled by the elongation factor-1α (EF-1α) promoter.

The packaging process of lentivirus was as follows:

(1) About 2.5×10⁶ 293T cells in good growth status (generally, cells with passage number less than 20 were selected) were added into 5 ml of 293T cell complete culture medium (high-sugar DMEM containing 10% FBS) and well mixed and then inoculated into a 6 cm culture dish (20-24 hours later, the cell confluence was required to reach 80-90%) and incubated overnight in a 37° C., 5% CO₂ incubator.

(2) About 20-24 hours later, a total of 6 μg of three plasmids (the mass ratio of CAR lentivirus core plasmid:psPAX2:PMD2.G=4:3:2) were added into 300 μL of Opti-MEM medium and mixed and 18 μL of a FuGENEHD transfection reagent (Promega, E2311) was then added and well mixed carefully. The mixed solution was set still at room temperature for 15-20 minutes, and then carefully added dropwise into a 6 cm culture dish and shaken carefully. The cells were incubated overnight in a 37° C., 5% CO₂ incubator.

(3) About 20-24 hours after transfection, the upper liquid was carefully removed from the 6 cm culture dish, and 5 mL of 293T cell complete culture medium was then added slowly.

(4) About 48 hours after transfection, the upper liquid (containing dead cells and debris) in the 6 cm culture dish was harvested and centrifuged at 3000 g for 5 minutes. The upper virus liquid with the cell debris removed was transferred to a new centrifuge tube, aliquoted and stored at −80° C. for later use.

### Example 4 Determination of Virus Titer

Lentivirus titer was determined as follows:

(1) The virus liquid prepared in Example 3 was thawed quickly.

(2) 293T cells in good growth status (generally, cells with passage number less than 20) were taken, the upper waste liquid was discarded, the cells were washed with PBS and digested with 0.25% trypsin (GIBICO) at 37° C. for about 3 minutes. After the cells were completely digested, a certain volume of 293T cell complete culture medium was added to stop the reaction, and samples were taken for cell counting. The cell density was then adjusted to 2.0×10⁵ cells/mL, and polybrene with a final concentration of 10 μg/mL was added, and then the cells were inoculated in six-well plates at 2.5 mL/well.

(3) The above virus liquid was then added to the six-well plates respectively. Generally, three loading gradients, i.e., 2 μL, 5 μL and 10 μL, are made for each virus liquid, and one to two blank wells without the virus liquid need to be set up as negative control for flow cytometry.

(4) The cell culture plates were placed in a 37° C., 5% CO₂ incubator for static culture.

(5) After the cells were cultured statically for 48 hours, the upper waste liquid was discarded. The cells were washed with PBS and then digested with 0.25% trypsin (GIBICO) at 37° C. for about 3 minutes. After the cells were completely digested, a certain volume of 293T cell complete culture medium was added to stop the reaction, and samples were taken for cell counting. About 5×10⁵ cells were then taken and placed in each 1.5 mL sterile centrifuge tube and marked.

(6) NBS solution (PBS solution containing 1% newborn calf serum) was added to make up to 1.2-1.5 mL. The mixed solution was then centrifuged at 4° C., 500 g for 5 minutes and the supernatant was then discarded.

(7) 1 mL of NBS was added into each tube, and the mixed solution was gently pipetted and well mixed, and then centrifuged at 4° C., 500 g for 5 minutes, and the supernatant was then discarded.

(8) 50 μl of NBS and 0.5 μg of BCMA-Fc-Avi protein (Acro, BC7-H82FO) were added into each tube. The mixed solution was gently pipetted with a pipette and well mixed (no primary antibody was added to the negative control), and incubated at 4° C. for 60 minutes.

(9) After incubation, 1 ml of NBS was directly added to resuspend the cells, the cell resuspension was then centrifuged at 4° C., 500 g for 5 minutes, and the supernatant was discarded.

(10) 50 μl of NBS and 0.5 μl of Native Streptavidin protein (DyLight® 650) (Abcam, ab134341) were added into each tube (no Native Streptavidin protein was added to the negative control), and then incubated at 4° C. for 30 minutes.

(11) Step (9) was performed again.

(12) 200 μl of NBS-resuspended cells were added into each tube.

(13) The fluorescence expression percentage was detected on a flow cytometer BD FACS CantoII, and the fluorescence was Dylight 650.

(14) Data processing: The results are usable only when the positive rate of test tube minus the positive rate of negative control is within a range of 5% to 20%.

Lentivirus titer (IU/mL)=number of cells on plate×(positive rate of test tube−positive rate of control tube)/volume of inoculated virus liquid

After testing, the titer of each of the above scFv-containing CAR viruses ranged from 5×10⁶ IU/mL to 15×10⁶ IU/mL.

### Example 5 Infection of T Cell and In Vitro Expansion of BCMA CAR-T Cells

The CAR-T cell containing the BCMA scFv sequence was prepared as follows:

(1) Human peripheral blood mononuclear cells (PBMC) were isolated, by Ficoll density gradient centrifugation, from blood samples collected by an apheresis machine (apheresis blood was provided by volunteers of the Ark Project initiated by Oricell Therapeutics Co., Ltd.).

(2) After PBMC was sorted by CD3-positive magnetic beads, CD3⁺T cells with a purity >90% were obtained. Please refer to the product manual (MACS, DS130-050-101) for the specific method of T sorting.

(3) The CD3⁺ T cells were resuspended with a T cell complete culture medium that was prepared by sequentially adding 5% FBS and 300 IU/mL interleukin 2 (125Ala) to the (X-VIVO 15 (Lonza) medium). Washed CD3/CD28 Dynabeads (Gibco, 40203D) were added at 3 times the amount of cells, T cell complete culture medium was added again, the cell density was adjusted to 1.0-1.2×10⁶ cells/mL, and the cells were placed in a 37° C., 5% CO₂ incubator for activation and culture (denoted as DO).

(4) Generally, lentiviral transduction was performed 20-24 hours after T cell activation.

(5) T cells that have been activated for 20-24 hours were collected, centrifuged at 500 g for 5 minutes, and resuspended in a certain volume of T cell complete culture medium and then sampled and counted. According to the ratio of the virus multiplicity of infection (MOI) of 3-6, the virus liquid having the titer determined in Example 3 was added respectively, and polybrene was then added till a final concentration of 5 μg/ml. Next, the T cell complete culture medium was added again, the cell density was adjusted to 0.6-1.0×10⁶ cells/mL, and the cells were then incubated at 37° C., 5% CO₂ incubator. Another portion of T cells without lentiviral infection was used as a negative control group.

(6) About 20-24 hours after viral transduction, T cells were collected from each group, centrifuged at 500 g for 5 minutes, resuspended in a certain volume of T cell complete medium, and then sampled and counted. The T cell complete culture medium was added again and the cell density was adjusted to 0.5-0.7×10⁶ cells/mL

(7) Then, CAR-T cells were counted everyday or every two days, and the T cell complete culture medium was added again according to the actual situation, and the cell density was adjusted to 0.5-1.0×10⁶ cells/mL.

(8) Generally, Dynabeads were removed on D7 of culture, and the total culture period was about 12 days.

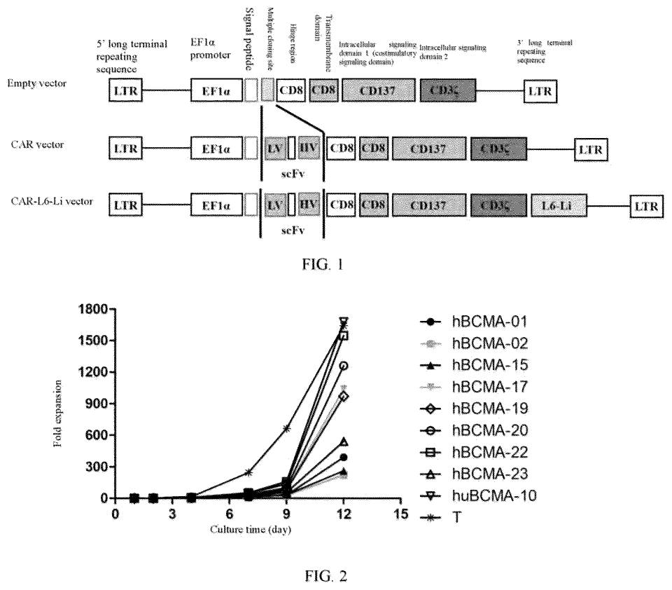

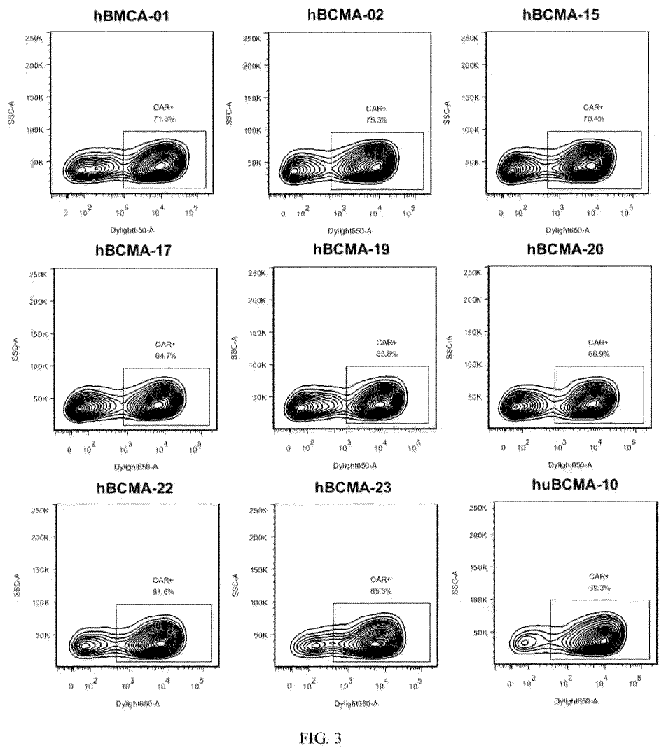

As shown in FIGS. 2 and 3, the total expansion fold of BCMACAR-T cells after activation and culture for 12 days ranges from 100 to 1600, and the CAR positivity rate after infection ranges from 65% to 90%. All can be used for cytological functional experiments.

### Example 6 Evaluation on In Vitro Targeted Killing Activity of CAR-T Cells

The MM1s cell is a myeloma cell that expresses BCMA, and the K562 cell is a tumor cell that does not express BCMA. In this test, we first constructed these two tumor cells that stably express Luciferase, and then co-incubated with the CAR-T cell for a period of time, and then the activity of the Luciferase enzyme was tested through the substrate on the basis of a principle which was detailed in Promega, E2510, to determine the targeted killing ability of the CAR-T cell.

MM1s-Luci and K562-Luci that overexpress Luciferase were constructed as follows:

(1) The pGL4.30 [luc2P/NFAT-RE/Hygro] Vector was purchased from Promega Company, the luc2P reporter gene (228-2003) in the vector was cloned into the lentivirus core empty plasmid without the CAR structure (the empty vector contains the GFP reporter gene), and the resulting plasmid was defined as Luciferase-GFP lentivirus core plasmid.

(2) The Luciferase-GFP (Luci-GFP) lentivirus was packaged according to the method stated in Example 3, and the titer of the Luciferase-GFP lentivirus was tested through a GFP fluorescence channel with reference to the method stated in Example 4.

(3) According to MOI=5-10, the MM1s cell and the K562 cell were infected respectively. After stable culture for 3-4 weeks, the GFP⁺ cell colonies were sorted through a flow sorter to construct MM1s-Luci-GFP cell and K562-Luci-GFP cell.

The in vitro killing activity of the CAR-T cell was tested during the culture period of D8 to D12. The CAR positivity rate of each group was required to be tested in advance and 0.5-1.0×10⁶ cells were taken. For the testing method, please refer to steps (6)-(13) in Example 4.

The cell infected with CAR lentivirus was an effector cell, the T cell not infected with lentivirus was a blank effector cell, and the MM1s-Luci-GFP cell and the K562-Luci-GFP were target cells.

The testing process of in vitro killing activity of CAR-T cell was as follows:

(1) The CAR positivity rate of each group was tested by flow cytometry one day in advance.

(2) On the day of the killing test, the CAR positive rates of all the groups were adjusted to be consistent by adding blank effector cells, and the medium was CAR-T medium without cytokines.

(3) Killing activity testing was carried out in 96-well plates. Three effector-target ratios of 3:1, 1:1 and 0.3:1 (or two effector-target ratios of 3:1 and 1:1) were set. The number of target cells in each 96-well plate was fixed at 2×10⁴. The target cell and the effector cell were added in sequence. Three replicate wells were set at each effector-to-target ratio for each group, and it was required to set another three separate target cell wells as negative controls.

(4) Generally, the killing activity was tested 5-6 hours after killing. That is, the cell suspension in the 96-well plates was first pipetted and well mixed, ½ volume of the cell suspension was taken evenly and added into completely blank 96-well plates, an equal volume of Luciferase substrate was then added and the cell solution was then kept at room temperature for 10-15 minutes in the dark. The corresponding fluorescence value was then read on the ELIASA. For the specific testing method, please refer to Steady-Glo® Luciferase Detection System (Promega, E2510).

(5) Since only viable MM1s cells can be detected, the killing toxicity of the CAR-T cell against the target cell can be calculated as follows:

$$Killing toxicity ( \% ) = \frac{\text{?}}{Fluorescence {value}_{\text{?}}} \times 100 \text{?}$$

$$\text{?} \text{indicates text missing or illegible when filed}$$

As shown in FIGS. 4A and 4B, compared with blank effector cell T cell, the BCMACAR-T cells of all groups have strong killing activity against BCMA-expressing MM1s cells and also show obvious dependence on the effector-target ratio, but have no killing effect on the K562 cell that does not express BCMA, proving that the tumor cell killing effect of CAR-T cells is specific. As can be seen from FIG. 4, at the three effector-to-target ratios, hBCMA-17, hBCMA-19, hBCMA-20, hBCMA-22, and hBCMA-23 of this application are all stronger than the positive control huBCMA-10. At the effector-to-target ratio of 1:1, the killing toxicity against the target cell is greater than 20%. At the effector-to-target ratio of 3:1, the killing toxicity against the target cell is greater than 70%.

### Example 7 IFN-γ Secretion after In Vitro Killing of BCMA CAR-T Cells

Referring to Example 6, the target cell killing experiment was carried out in 96-well plates. The effector-target ratio was set at 1:3. The number of effector cells in each 96-well plate was fixed at 2×10⁴, and the target cell and the effector cell were then added in sequence. Three replicate wells were set at each effector-to-target ratio for each group, and it was required to set three separate effector cell wells for each group. The background factor secretion of the effector cell was then tested.

About 24 hours after the target cell was killed, the cell solution in the 96-well plate was centrifuged and the supernatant was collected from each well (if not tested immediately, the supernatant was required to be stored in a −80° C. refrigerator).

IFN-γ cytokine secretion (R&D, DY285) was tested by the Elisa method as follows:

(1) The IFN-γ capture antibody in the kit was added into a 96-well plate in advance and the plate was coated overnight at room temperature.

(2) The coated 96-well plate was washed with a buffer, a blocking solution was added for blocking at room temperature for 1 hour, and then the 96-well plate was washed with a buffer and set aside for later use.

(3) According to the instructions, the standard product in the kit was gradient diluted, and in the meanwhile the collected supernatant to be tested was diluted by 5-30 folds. The diluted standard product and the sample to be tested were then added into the 96-well plate treated in the above steps, and incubation was then carried out at room temperature for 2 hours.

(4) After all the wells were washed with a buffer, the IFN-γ test antibody in the kit was added, and incubation was then carried out at room temperature for 2 hours.

(5) After all the wells were washed with a buffer, Streptavidin-HRP in the kit was added and incubation was then carried out at room temperature in the dark for 20 minutes.

(6) After all the wells were washed with a buffer, a substrate solution TMB (for its formula, please refer to R&D, DY285) was added and incubation was then carried out at room temperature in the dark for 20 minutes.

(7) A stop buffer (for its formula, please refer to R&D, DY285) was added, and the value corresponding to OD450 was read on the ELIASA.

For the working concentrations of various reagents, including IFN-γ standard product, capture antibody, test antibody, Streptavidin-HRP, please refer to the instructions (R&D, DY285) for details. For the formulas of other reagents, including the buffer, the blocking solution, the diluent, the substrate solution, and the stop buffer, please refer to R&D, DY285 for details.

Finally, the standard curves and the corresponding fitting formula were obtained according to the four-parameter fitting method. From the fitting formula and the dilution factor of the sample to be tested, the concentration of IFN-γ in the sample to be tested was calculated.

As shown in FIG. 5, without stimulation of the MM1s cell, CAR-T cells of all groups did not secrete IFN-γ. Under the stimulation of the MM1s cell, compared with the positive control huBCMA-10 (Patent Application No. US 2017/0226216 A1) selected in this application, the hBCMA-17, hBCMA-19, hBCMA-20, hBCMA-22, and hBCMA-23 of this patent application can secrete more IFN-γ when killing the target cell, with the secretion amount of 1500 μg/mL or above, even up to 2000 μg/mL.

### Example 8 Targeted Proliferation Ability of BCMA CAR-T Cells

Since the survival rate of the MM1s cell after γ-ray irradiation is extremely low, untreated MM1s cell was selected as a target cell for testing the targeted proliferation ability of CAR-T cells in this example.

Generally, the testing is carried out after the CAR-T cell is cultured for about 9-12 days. The CAR positive rate of each group needs to be detected in advance, and then blank effector T cell is used to adjust the CAR positive rates of all the groups to be consistent (the culture medium is X-VIVO15 medium without any additives).

A CAR-T cell and the MM1s cell are subjected to the first round of targeted stimulation at an effector-to-target ratio of 1:1. After co-incubation for 4-5 days, all cells are collected, sampled and counted. The second round of targeted stimulation is then carried out at a ratio of 0.5:1 (total CAR-T cell:new MM1s cell), and after another 4-5 days of co-incubation, the third round of targeted stimulation, where the ratio of the two cells is the same as that in the second round of targeted stimulation). During the three rounds of targeted stimulation, an appropriate amount of X-VIVO15 culture medium is added everyday or every two days according to the cell growth condition.

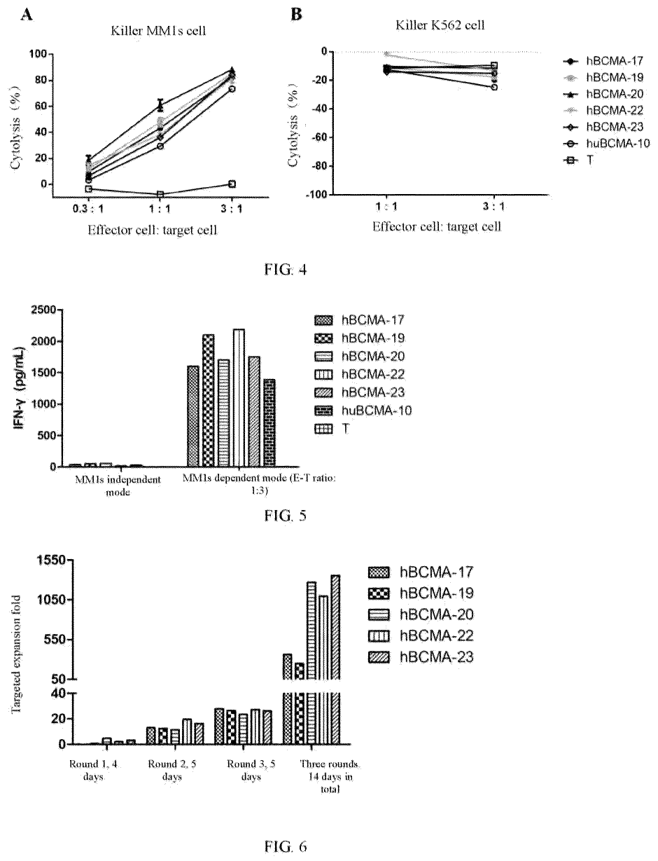

As shown in FIG. 6, after three rounds of targeted stimulation of the BCMACAR-T cells, the targeted expansion folds of the hBCMA-17, hBCMA-19, hBCMA-20, hBCMA-22, and hBCMA-23 of this application are all high.

### Example 9 In Vitro Targeted Killing Activity of BCMA CAR-T Cells Based on the L6-Li CAR Structure and IFNγ Secretion after Killing

As described in Example 2, the nucleic acid sequences of scFv fragments of hBCMA-22 and hu-BCMA-10 were respectively constructed into a CAR lentivirus vector containing L6-Li to obtain the lentivirus vectors of hBCMA-22-L6-Li and hu-BCMA-10-L6-Li. After lentivirus packaging and virus titer determination, and in vitro expansion of CAR-T cells, the in vitro killing activity of CAR-T cells and IFN-γ secretion after killing were tested during the period of D8 to D12. For specific operation methods, please refer to Examples 3-7.

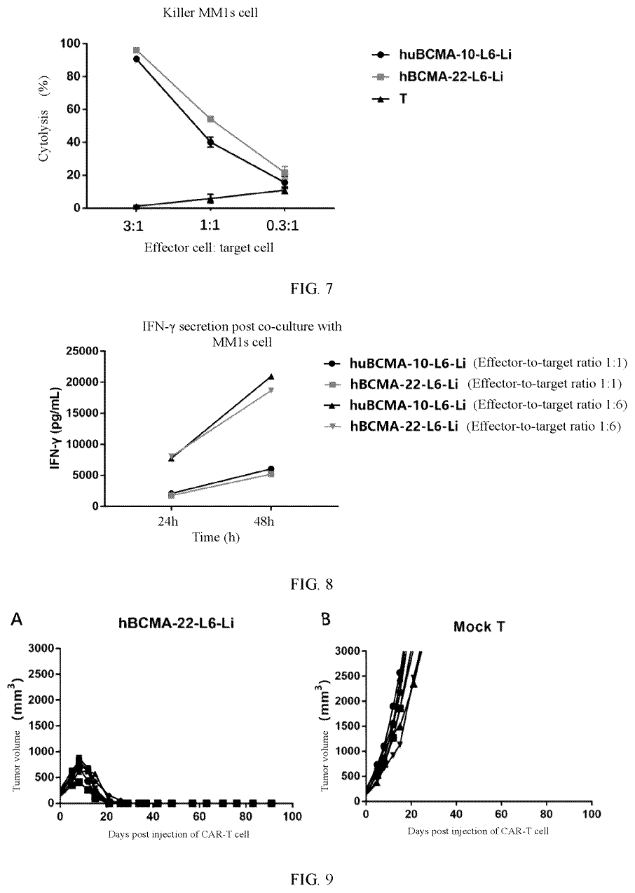

As shown in FIG. 7, the specific killing abilities of CAR-T cells based on hBCMA-22-L6-Li and hu-BCMA-10-L6-Li against MM1s cell at three effector-to-target ratios are basically the same. At the effector-target ratio of 3:1, the killing toxicity of CAR-T cells against the target cell is about 90%. At the effector-target ratio of 1:1, the killing toxicity of CAR-T cells is about 40%. As shown in FIG. 8, the IFN-γ secretion abilities of these two groups of CAR-T cells after co-culture with the MM1s cell are basically consistent. At the effector-to-target ratio of 1:1, CAR-T cells and the target cell can stimulate the secretion of IFN-γ to approximately 5,000 μg/mL after co-culture for 48 hours. At the effector-to-target ratio of 1:6, CAR-T cells and the target cell can stimulate the secretion of IFN-γ to approximately 17500 pg/mL after co-culture for 48 hours.

### Example 10 Tumor Inhibition and Decreasing Ability of BCMA CAR-T Cells Based on the hBCMA-22-L6-Li CAR Structure in Tumor-Bearing Mice and their Metabolism in Mice

6-week-old mice were prepared and each of the mice was inoculated with 1×10⁷ MM1s cells subcutaneously. 14 days later, the volumes of the subcutaneous tumors in the mice were about within a range of 150 mm³ to 250 mm³. The mice are divided into two groups (eight mice in each group) and inoculated with freshly prepared CAR-T cells (2×10⁶ CAR⁺/mouse) in the hBCMA-22-L6-Li group and negative control T cells (the number of cells inoculated per mouse in the T cell group was consistent with the total number of cells corresponding to 2×10⁶ CAR⁺ in the hBCMA-22-L6-Li group), and defined as D0 after CAR-T injection.

After inoculation with CAR-T cells, twice a week, the tumor size was measured with a vernier caliper, the body weight of the mice was weighed, and the hair color, excrement, food and water intake, body movement, and death of mice were observed. On D7, D14, and D21 after CAR-T injection, blood was collected from the tail vein of 5 mice in each group to detect cytokine secretion (IFN-γ and IL-2 cytokines were detected with Th1/2/17 CBA) and the proportion of human T cells (hCD3 and hCD8). The mice were sacrificed when their tumor volumes were greater than 3000 mm³, and all other mice were sacrificed on D90 to end the experiment.

10.1 Detection of hCD3 and hCD8 Molecular Markers in Peripheral Blood of Mice by Flow Cytometry
(1) 500 ul of PBS was added into a 10 mL human EDTA.K2 blood collection tube (purchased from Jiangsu Kangjie Medical Devices) and well mixed, and 20 ul was then taken from the blood collection tube and placed into a corresponding 1.5 ml EP tube;
(2) 30 μl of blood was collected from the tail vein of the mice, and then there was 50 μl of solution in total;
(3) The solution was centrifuged at 3000 rpm for 10 minutes, and 15 μl of the supernatant was taken out for testing CBA (if not tested immediately, it must be stored in a −80° C. refrigerator);
(4) 40 ul of PBS was added to the remaining 35 μl of solution, and the solution was well mixed again and then transferred to a flow cytometer tube;
(5) APC-Anti-Human-CD3 antibody (BD, 555335) and Percp-cy5.5-Anti-Human-CD8 antibody (BD, 341051) were added;
(6) The solution was well mixed with a vortexer at a low speed and then incubated in a 4° C. refrigerator for 30 minutes;
(7) 2 ml of red blood cell lysis buffer (BD, 34902) was directly added without washing, and the solution was well mixed with a vortexer at a low speed, and then incubated at room temperature in the dark for 10 minutes;
(8) The solution was centrifuged at 500 g for 4 minutes and the centrifuge tube was then placed upside down to discard the supernatant;
(9) 2 ml of the NBS solution in Example 4 was added and the mixed solution was then well mixed with a vortexer at a low speed;
(10) The solution was centrifuged at 500 g for 4 minutes; and
(11) The cell pellet was resuspended in 200 ul of NBS solution and cell resuspension was then well mixed for flow cytometry.
   10.2 Method for Detecting Cytokines (IFN-γ and IL-2) with TH1/2/17 CBA (BD, 560484)
(1) Based on the number of samples, the number of beads required was calculated;
(2) Each captured magnetic bead suspension tube (A1-A7) was vortexed vigorously for at least 1 minute;
(3) A 1.5 ml EP tube was used and denoted as TH1/2/17 beads;
(4) The required number of A1-A7 beads were placed in the EP tube;
(5) The mixed beads suspension was vortexed for 1 minute and well mixed;
(6) The suspension was centrifuged at 300 g for 5 minutes;
(7) The supernatant was removed and the TH1/2/17 beads were resuspended in an equal volume of Serum Enhancement Buffer (included in the kit);
(8) After vortexing for 1 minute, the cells were incubated at room temperature in the dark for 30 minutes;
(9) 50 μl of plasma samples of mice were added into a new EP tube, and then 50 μl of TH1/2/17 beads and 50 μl of PE-detection Regent (included in the kit) were added in sequence,
(10) After vortexing for 30s, the cells were incubated at room temperature in the dark for 3 hours;
(11) 500 ul of PBS was added and the solution was well vortexed and then centrifuged at 300 g for 5 minutes; and
(12) The supernatant was discarded, resuspension was carried out in 200 μl of PBS, and the solution was well vortexed and then transferred to a flow cytometer tube for flow cytometer.

As shown in FIGS. 9A and 9B, after the CAR-T cells of the hBCMA-22-L6-Li group were injected into tumor-bearing mice, the tumor volume continued to increase and reached the maximum at D8, which was close to the average tumor volume of the Mock T group at this time. In about two weeks, the tumors of the hBCMA-22-L6-Li group began to decrease, and the tumors completely disappeared in about four weeks. In the third week, the tumor volume of the Mock T group had increased to 3000 mm³ or larger.

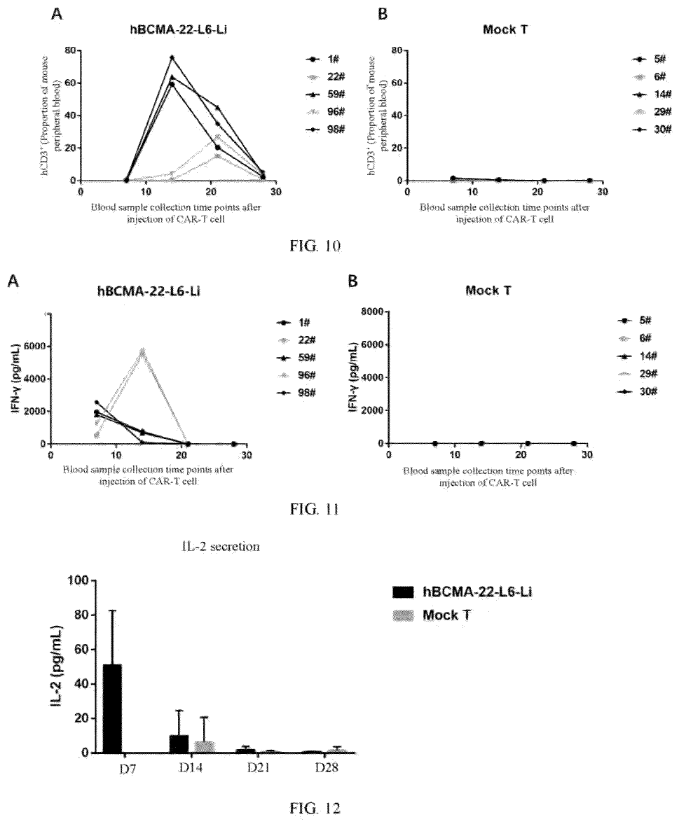

FIGS. 10A and 10B show the proliferation and metabolism of CAR-T cells of the hBCMA-22-L6-Li group in tumor-bearing mice (taking hCD3+ cells as an example, the changing trend of the proportion of hCD8+ cells is basically consistent with that of hCD3+, and no data is shown). The starting point and peak point of rapid expansion of CAR-T cells in different mice are not the same (the peak point of hCD3 may appear between D14 and D21). On the average, the average hCD3⁺T cell content is about 40% on D14 of CAR-T injection and then gradually decreases. On D28, the average hCD3⁺T cell content drops to 5% or below. No T cell proliferation in the Mock T group was detected at four detection time points.

While inhibiting and decreasing tumors, a large amount of IFN-γ and a small amount of IL-2 cytokines were detected in the peripheral blood of mice (the secretion of IL-4, IL-6, IL-10, TNF-α, and IL17A cytokines was not detected and no data is shown). As shown in FIG. 11A, the starting point and peak point of IFN-γ cytokine secretion in different mice are also different, and with reference to FIG. 10A, it can be seen that the peaks of IFN-γ cytokine secretion are all earlier than the proliferation peak of CAR-T cell, and the IFN-γ cytokine content drops to 3 pg/ml or below on D21. IFN-γ secretion in the Mock T group is not detected at four detection time points.

The secretion level of IL-2 cytokines is significantly lower than that of IFN-γ (FIG. 12) and reaches a peak (50 pg/ml on average) on D7 of CAR-T injection, and then declines rapidly.

The aforementioned details are provided by way of explanations and examples and are not intended to limit the scope of the appended claims. At present, the various changes in the embodiments stated in this application are obvious to those of ordinary skills in the art and fall within the scope of the appended claims and their equivalent solutions.

1. A chimeric antigen receptor (CAR), comprising a BCMA (B cell maturation antigen) binding domain,
said BCMA binding domain comprising an antibody or an antigen-binding fragment of an antibody that specifically binds to BCMA,
wherein said antibody comprises a heavy-chain complementarity determining region 3 (HCDR3), a heavy-chain complementarity determining region 2 (HCDR2), and a heavy-chain complementarity determining region 1 (HCDR1), wherein said HCDR3 comprises an amino acid sequence as set forth in SEQ ID NO: 25, said HCDR2 comprises an amino acid sequence as set forth in SEQ ID NO: 41, and said HCDR1 comprises an amino acid sequence as set forth in SEQ ID NO: 21.

2. (canceled)

3. The CAR of claim 1, wherein said HCDR2 comprises an amino acid sequence as set forth in any one of SEQ ID NOs: 22-24.

4-6. (canceled)

7. The CAR of claim 1, wherein said antibody comprises any set of HCDR1, HCDR2, and HCDR3, selected from the groups consisting of:
(1) HCDR1: SEQ ID NO: 21, HCDR2: SEQ ID NO: 22, and HCDR3: SEQ ID NO: 25;
(2) HCDR1: SEQ ID NO: 21, HCDR2: SEQ ID NO: 23, and HCDR3: SEQ ID NO: 25; and
(3) HCDR1: SEQ ID NO: 21, HCDR2: SEQ ID NO: 24, and HCDR3: SEQ ID NO: 25.

8-17. (canceled)

18. The CAR of claim 1, wherein said antibody comprises a heavy chain variable region (VH), and said VH comprises an amino acid sequence as set forth in SEQ ID NO: 46.

19. The CAR of claim 18, wherein said VH comprises an amino acid sequence as set forth in any one of SEQ ID NOs: 5-9.

20. The CAR of claim 1, wherein said antibody comprises a light-chain complementarity determining region 3 (LCDR3), a light-chain complementarity determining region 2 (LCDR2), and a light-chain complementarity determining region 1 (LCDR1), wherein said LCDR3 comprises an amino acid sequence as set forth in SEQ ID NO: 40, said LCDR2 comprises an amino acid sequence as set forth in SEQ ID NO: 39, and said LCDR1 comprises an amino acid sequence as set forth in SEQ ID NO: 38.

21-26. (canceled)

27. The CAR of claim 20 wherein said LCDR1 comprises an amino acid sequence as set forth in any one of SEQ ID NOs: 10-12, said LCDR2 comprises an amino acid sequence as set forth in any one of SEQ ID NO: 13-14, and said LCDR3 comprises an amino acid sequence as set forth in any one of SEQ ID NO: 15-16.

28. The CAR of claim 20, wherein said antibody comprises any set of LCDR1, LCDR2, and LCDR3, selected from the groups consisting of:
(1) LCDR1: SEQ ID NO: 10, LCDR2: SEQ ID NO: 13, and LCDR3: SEQ ID NO: 15;
(2) LCDR1: SEQ ID NO: 11, LCDR2: SEQ ID NO: 13, and LCDR3: SEQ ID NO: 16;
(3) LCDR1: SEQ ID NO: 12, LCDR2: SEQ ID NO: 13, and LCDR3: SEQ ID NO: 16; and
(4) LCDR1: SEQ ID NO: 12, LCDR2: SEQ ID NO: 14, and LCDR3: SEQ ID NO: 16.

29-33. (canceled)

34. The CAR of claim 1, wherein said antibody comprises a light chain variable region (VL), and said VL comprises an amino acid sequence as set forth in SEQ ID NO: 45.

35. The CAR of claim 34, wherein said VL comprises an amino acid sequence as set forth in any one of SEQ ID NOs: 1-4.

36-37. (canceled)

38. The CAR of claim 1, wherein said antibody comprises a VH and a VL, and said VH and VL comprises any set of amino acid sequence, selected from the groups consisting of:
(1) VH: SEQ ID NO: 5 and VL: SEQ ID NO: 1;
(2) VH: SEQ ID NO: 6 and VL: SEQ ID NO: 2;
(3) VH: SEQ ID NO: 7 and VL: SEQ ID NO: 2;
(4) VH: SEQ ID NO: 8 and VL: SEQ ID NO: 2;
(5) VH: SEQ ID NO: 9 and VL: SEQ ID NO: 3;
(6) VH: SEQ ID NO: 8 and VL: SEQ ID NO: 3;
(7) VH: SEQ ID NO: 9 and VL: SEQ ID NO: 4; and
(8) VH: SEQ ID NO: 8 and VL: SEQ ID NO: 4.

39. The CAR of claim 1, wherein said BCMA binding domain comprises a scFv, and said scFv comprises a VH and a VL of said antibody that specifically binds to BCMA, wherein said scFv comprises an amino acid sequence as set forth in any one of SEQ ID NOs: 49-56.

40-43. (canceled)

44. The CAR of claim 1, comprising a transmembrane domain, a costimulatory domain, an intracellular signaling domain, a hinge region and a signal peptide,
wherein said transmembrane domain comprises transmembrane domains derived from proteins selected from the group consisting of: CD28, CD3e, CD45, CD4, CD5, CD8, CD9, CD16, CD22, CD33, CD37, CD64, CD80, CD86, CD134, CD137, and CD154,
wherein said costimulatory domain comprises costimulatory domains derived from proteins selected from the group consisting of: CD137, CD28, 4-1BB, OX-40, and ICOS,
wherein said intracellular signaling domain comprises an intracellular signaling domain derived from CD3ζ,
wherein said hinge region comprises a hinge region derived from CD8, and
wherein said signal peptide comprises an amino acid sequence as set forth in SEQ ID NO: 59.

45. The CAR of claim 44, wherein said transmembrane domain is derived from CD8, and said transmembrane domain comprises an amino acid sequence as set forth in SEQ ID NO: 61,
said costimulatory domain is derived from CD137, and said costimulatory domain comprises an amino acid sequence as set forth in SEQ ID NO: 62,
said intracellular signaling domain comprises an amino acid sequence as set forth in SEQ ID NO: 63,
said hinge region is derived from CD8 and comprises an amino acid sequence as set forth in SEQ ID NO: 60, and
said signal peptide comprises an amino acid sequence as set forth in SEQ ID NO: 59.

46-53. (canceled)

54. The CAR of claim 44, comprising L6-Li, wherein said L6-Li is located at a C terminal of said intracellular signaling domain and said L6-Li comprises an amino acid sequence as set forth in SEQ ID NO: 107.

55. (canceled)

56. The CAR of claim 1, comprising an amino acid sequence as set forth in any one of SEQ ID NOs: 64-71 and 74.

57. (canceled)

58. An isolated nucleic acid molecule, comprising a nucleotide sequence encoding the CAR of claim 1.

59-64. (canceled)

65. An immune effector cell, comprising the CAR of claim 1.

66-67. (canceled)

68. A pharmaceutical composition, comprising the CAR of claim 1 and a pharmaceutically acceptable carrier.

69-74. (canceled)

75. A method for treating a disease or disorder associated with BCMA expression, comprising: administering the CAR of claim 1 to a subject in need.

76-86. (canceled)


SEQUENCE LISTING (107 sequences)

SEQ ID NO: 1 - protein, 111 aa, Artificial Sequence
  VL
EIVLTQSPATLSLSPGERATLSCRASESVSVIGAHLIHWYQQKPGQAPRLLIYLGRNRAAGIPARFSGSGSGTDFTLTISSLEPEDAAIYYCLQSRIFPRTFGQGTKLEIK

SEQ ID NO: 2 - protein, 111 aa, Artificial Sequence
  VL
EIVLTQSPATLSLSPGERATLSCRASESVSVSGIHLLHWYQQKPGQAPRLLIYLGRNRAAGIPARFSGSGSGTDFTLTISSLEPEDAAIYYCLQSRRFPRHFGQGTKLEIK

SEQ ID NO: 3 - protein, 111 aa, Artificial Sequence
  VL
EIVLTQSPATLSLSPGERATLSCRASESVSVPGISFIHWYQQKPGQAPRLLIYLGRNRAAGIPARFSGSGSGTDFTLTISSLEPEDAAIYYCLQSRRFPRHFGQGTKLEIK

SEQ ID NO: 4 - protein, 111 aa, Artificial Sequence
  VL
EIVLTQSPATLSLSPGERATLSCRASESVSVPGISFIHWYQQKPGQAPRLLIYLGSNRPSGIPARFSGSGSGTDFTLTISSLEPEDAAIYYCLQSRRFPRHFGQGTKLEIK

SEQ ID NO: 5 - protein, 117 aa, Artificial Sequence
  VH
QVQLVQSGSELKKPGASVKVSCKASGYTFADYTINWVRQATGQGLEWMGWINTETREPAYAYDFRGRFVFSLDTSVSTAYLQISSLKAEDTAVYYCARDIRYVMDYWGQGTLVTVSS

SEQ ID NO: 6 - protein, 117 aa, Artificial Sequence
  VH
QVQLVQSGSELKKPGASVKVSCKASGYSFADYTINWVRQAPGQGLEWIGWINTETREPAYAYDFRSRFVFSLDTSASTAYLQISSLKAEDTAVYYCARDIRYVMDYWGQGTLVTVSS

SEQ ID NO: 7 - protein, 117 aa, Artificial Sequence
  VH
QVQLVQSGSEVKQPGASVKVSCKASGYSFADYTINWVRQAPGQGLEWMGWINTETREPTYAYDFRSRFVFSLDTSASTAYLELSSLRSEDTAVYYCARDIRYVMDYWGQGTLVTVSS

SEQ ID NO: 8 - protein, 117 aa, Artificial Sequence
  VH
QVQLVQSGAEVKKPGASVKVSCKASGYTFADYTINWVRQATGQGLEWMGWINTETREPTYAYDFRSRFTFTLNTSISTAYLELSSLRSEDTAVYYCARDIRYVMDYWGQGTLVTVSS

SEQ ID NO: 9 - protein, 117 aa, Artificial Sequence
  VH
QVQLVQSGAEVKKPGASVKVSCKASGYTFADYTINWVRQATGQGLEWMGWINTETREPAYAYDFRGRFTFTLNTSISTAYLELSSLRSEDTAVYYCARDIRYVMDYWGQGTLVTVSS

SEQ ID NO: 10 - protein, 15 aa, Artificial Sequence
  LCDR1
RASESVSVIGAHLIH

SEQ ID NO: 11 - protein, 15 aa, Artificial Sequence
  LCDR1
RASESVSVSGIHLLH

SEQ ID NO: 12 - protein, 15 aa, Artificial Sequence
  LCDR1
RASESVSVPGISFIH

SEQ ID NO: 13 - protein, 7 aa, Artificial Sequence
  LCDR2
LGRNRAA

SEQ ID NO: 14 - protein, 7 aa, Artificial Sequence
  LCDR2
LGSNRPS

SEQ ID NO: 15 - protein, 9 aa, Artificial Sequence
  LCDR3
LQSRIFPRT

SEQ ID NO: 16 - protein, 9 aa, Artificial Sequence
  LCDR3
LQSRRFPRH

SEQ ID NO: 17 - protein, 23 aa, Artificial Sequence
  L-FR1
EIVLTQSPATLSLSPGERATLSC

SEQ ID NO: 18 - protein, 15 aa, Artificial Sequence
  L-FR2
WYQQKPGQAPRLLIY

SEQ ID NO: 19 - protein, 32 aa, Artificial Sequence
  L-FR3
GIPARFSGSGSGTDFTLTISSLEPEDAAIYYC

SEQ ID NO: 20 - protein, 10 aa, Artificial Sequence
  L-FR4
FGQGTKLEIK

SEQ ID NO: 21 - protein, 5 aa, Artificial Sequence
  HCDR1
DYTIN

SEQ ID NO: 22 - protein, 17 aa, Artificial Sequence
  HCDR2
WINTETREPAYAYDFRG

SEQ ID NO: 23 - protein, 17 aa, Artificial Sequence
  HCDR2
WINTETREPAYAYDFRS

SEQ ID NO: 24 - protein, 17 aa, Artificial Sequence
  HCDR2
WINTETREPTYAYDFRS

SEQ ID NO: 25 - protein, 8 aa, Artificial Sequence
  HCDR3
DIRYVMDY

SEQ ID NO: 26 - protein, 30 aa, Artificial Sequence
  H-FR1
QVQLVQSGSELKKPGASVKVSCKASGYTFA

SEQ ID NO: 27 - protein, 30 aa, Artificial Sequence
  H-FR1
QVQLVQSGSELKKPGASVKVSCKASGYSFA

SEQ ID NO: 28 - protein, 30 aa, Artificial Sequence
  H-FR1
QVQLVQSGSEVKQPGASVKVSCKASGYSFA

SEQ ID NO: 29 - protein, 30 aa, Artificial Sequence
  H-FR1
QVQLVQSGAEVKKPGASVKVSCKASGYTFA

SEQ ID NO: 30 - protein, 14 aa, Artificial Sequence
  H-FR2
WVRQATGQGLEWMG

SEQ ID NO: 31 - protein, 14 aa, Artificial Sequence
  H-FR2
WVRQAPGQGLEWIG

SEQ ID NO: 32 - protein, 14 aa, Artificial Sequence
  H-FR2
WVRQAPGQGLEWMG

SEQ ID NO: 33 - protein, 32 aa, Artificial Sequence
  H-FR3
RFVFSLDTSVSTAYLQISSLKAEDTAVYYCAR

SEQ ID NO: 34 - protein, 32 aa, Artificial Sequence
  H-FR3
RFVFSLDTSASTAYLQISSLKAEDTAVYYCAR

SEQ ID NO: 35 - protein, 32 aa, Artificial Sequence
  H-FR3
RFVFSLDTSASTAYLELSSLRSEDTAVYYCAR

SEQ ID NO: 36 - protein, 32 aa, Artificial Sequence
  H-FR3
RFTFTLNTSISTAYLELSSLRSEDTAVYYCAR

SEQ ID NO: 37 - protein, 11 aa, Artificial Sequence
  H-FR4
WGQGTLVTVSS

SEQ ID NO: 38 - protein, 15 aa, Artificial Sequence
  LCDR1
  misc_feature (9)..(9): Xaa = Ile Pro or Ser
  misc_feature (11)..(11): Xaa = Ala or Ile
  misc_feature (12)..(12): Xaa = His or Ser
  misc_feature (13)..(13): Xaa = Phe or Leu
  misc_feature (14)..(14): Xaa = Ile or Leu
RASESVSVXGXXXXH

SEQ ID NO: 39 - protein, 7 aa, Artificial Sequence
  LCDR2
  misc_feature (3)..(3): Xaa = Arg or Ser
  misc_feature (6)..(6): Xaa = Ala or Pro
  misc_feature (7)..(7): Xaa = Ala or Ser
LGXNRXX

SEQ ID NO: 40 - protein, 9 aa, Artificial Sequence
  LCDR3
  misc_feature (5)..(5): Xaa = Ile or Arg
  misc_feature (9)..(9): Xaa = His or Thr
LQSRXFPRX

SEQ ID NO: 41 - protein, 17 aa, Artificial Sequence
  HCDR2
  misc_feature (10)..(10): Xaa = Ala or Thr
  misc_feature (17)..(17): Xaa = Gly or Ser
WINTETREPXYAYDFRX

SEQ ID NO: 42 - protein, 30 aa, Artificial Sequence
  H-FR1
  misc_feature (9)..(9): Xaa = Ala or Ser
  misc_feature (11)..(11): Xaa = Leu or Val
  misc_feature (13)..(13): Xaa = Lys or Gln
  misc_feature (28)..(28): Xaa = Ser or Thr
QVQLVQSGXEXKXPGASVKVSCKASGYXFA

SEQ ID NO: 43 - protein, 14 aa, Artificial Sequence
  H-FR2
  misc_feature (6)..(6): Xaa = Pro or Thr
  misc_feature (13)..(13): Xaa = Ile or Met
WVRQAXGQGLEWXG

SEQ ID NO: 44 - protein, 32 aa, Artificial Sequence
  H-FR3
  misc_feature (3)..(3): Xaa = Thr or Val
  misc_feature (5)..(5): Xaa = Ser or Thr
  misc_feature (7)..(7): Xaa = Asp or Asn
  misc_feature (10)..(10): Xaa = Ala Ile or Val
  misc_feature (16)..(16): Xaa = Glu or Gln
  misc_feature (17)..(17): Xaa = Ile or Leu
  misc_feature (21)..(21): Xaa = Lys or Arg
  misc_feature (22)..(22): Xaa = Ala or Ser
RFXFXLXTSXSTAYLXXSSLXXEDTAVYYCAR

SEQ ID NO: 45 - protein, 111 aa, Artificial Sequence
  VL
  misc_feature (32)..(32): Xaa = Ile Pro or Ser
  misc_feature (34)..(34): Xaa = Ala or Ile
  misc_feature (35)..(35): Xaa = His or Ser
  misc_feature (36)..(36): Xaa = Phe or Leu
  misc_feature (37)..(37): Xaa = Ile or Leu
  misc_feature (56)..(56): Xaa = Arg or Ser
  misc_feature (59)..(59): Xaa = Ala or Pro
  misc_feature (60)..(60): Xaa = Ala or Ser
  misc_feature (97)..(97): Xaa = Ile or Arg
  misc_feature (101)..(101): Xaa = His or Thr
EIVLTQSPATLSLSPGERATLSCRASESVSVXGXXXXHWYQQKPGQAPRLLIYLGXNRXXGIPARFSGSGSGTDFTLTISSLEPEDAAIYYCLQSRXFPRXFGQGTKLEIK

SEQ ID NO: 46 - protein, 117 aa, Artificial Sequence
  VH
  misc_feature (9)..(9): Xaa = Ala or Ser
  misc_feature (11)..(11): Xaa = Leu or Val
  misc_feature (13)..(13): Xaa = Lys or Gln
  misc_feature (28)..(28): Xaa = Ser or Thr
  misc_feature (41)..(41): Xaa = Pro or Thr
  misc_feature (48)..(48): Xaa = Ile or Met
  misc_feature (59)..(59): Xaa = Ala or Thr
  misc_feature (66)..(66): Xaa = Gly or Ser
  misc_feature (69)..(69): Xaa = Thr or Val
  misc_feature (71)..(71): Xaa = Ser or Thr
  misc_feature (73)..(73): Xaa = Asp or Asn
  misc_feature (76)..(76): Xaa = Ala Ile or Val
  misc_feature (82)..(82): Xaa = Glu or Gln
  misc_feature (83)..(83): Xaa = Ile or Leu
  misc_feature (87)..(87): Xaa = Lys or Arg
  misc_feature (88)..(88): Xaa = Ala or Ser
QVQLVQSGXEXKXPGASVKVSCKASGYXFADYTINWVRQAXGQGLEWXGWINTETREPXYAYDFRXRFXFXLXTSXSTAYLXXSSLXXEDTAVYYCARDIRYVMDYWGQGTLVTVSS

SEQ ID NO: 47 - protein, 111 aa, Artificial Sequence
  VL
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIK

SEQ ID NO: 48 - protein, 117 aa, Artificial Sequence
  VH
QVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSS

SEQ ID NO: 49 - protein, 246 aa, Artificial Sequence
  hBCMA-01 scFv
EIVLTQSPATLSLSPGERATLSCRASESVSVIGAHLIHWYQQKPGQAPRLLIYLGRNRAAGIPARFSGSGSGTDFTLTISSLEPEDAAIYYCLQSRIFPRTFGQGTKLEIKGSTSGSGKPGSGEGSTKGQVQLVQSGSELKKPGASVKVSCKASGYTFADYTINWVRQATGQGLEWMGWINTETREPAYAYDFRGRFVFSLDTSVSTAYLQISSLKAEDTAVYYCARDIRYVMDYWGQGTLVTVSS

SEQ ID NO: 50 - protein, 246 aa, Artificial Sequence
  hBCMA-02 scFv
EIVLTQSPATLSLSPGERATLSCRASESVSVSGIHLLHWYQQKPGQAPRLLIYLGRNRAAGIPARFSGSGSGTDFTLTISSLEPEDAAIYYCLQSRRFPRHFGQGTKLEIKGSTSGSGKPGSGEGSTKGQVQLVQSGSELKKPGASVKVSCKASGYSFADYTINWVRQAPGQGLEWIGWINTETREPAYAYDFRSRFVFSLDTSASTAYLQISSLKAEDTAVYYCARDIRYVMDYWGQGTLVTVSS

SEQ ID NO: 51 - protein, 246 aa, Artificial Sequence
  hBCMA-15 scFv
EIVLTQSPATLSLSPGERATLSCRASESVSVSGIHLLHWYQQKPGQAPRLLIYLGRNRAAGIPARFSGSGSGTDFTLTISSLEPEDAAIYYCLQSRRFPRHFGQGTKLEIKGSTSGSGKPGSGEGSTKGQVQLVQSGSEVKQPGASVKVSCKASGYSFADYTINWVRQAPGQGLEWMGWINTETREPTYAYDFRSRFVFSLDTSASTAYLELSSLRSEDTAVYYCARDIRYVMDYWGQGTLVTVSS

SEQ ID NO: 52 - protein, 246 aa, Artificial Sequence
  hBCMA-17 scFv
EIVLTQSPATLSLSPGERATLSCRASESVSVSGIHLLHWYQQKPGQAPRLLIYLGRNRAAGIPARFSGSGSGTDFTLTISSLEPEDAAIYYCLQSRRFPRHFGQGTKLEIKGSTSGSGKPGSGEGSTKGQVQLVQSGAEVKKPGASVKVSCKASGYTFADYTINWVRQATGQGLEWMGWINTETREPTYAYDFRSRFTFTLNTSISTAYLELSSLRSEDTAVYYCARDIRYVMDYWGQGTLVTVSS

SEQ ID NO: 53 - protein, 246 aa, Artificial Sequence
  hBCMA-19 scFv
EIVLTQSPATLSLSPGERATLSCRASESVSVPGISFIHWYQQKPGQAPRLLIYLGRNRAAGIPARFSGSGSGTDFTLTISSLEPEDAAIYYCLQSRRFPRHFGQGTKLEIKGSTSGSGKPGSGEGSTKGQVQLVQSGAEVKKPGASVKVSCKASGYTFADYTINWVRQATGQGLEWMGWINTETREPAYAYDFRGRFTFTLNTSISTAYLELSSLRSEDTAVYYCARDIRYVMDYWGQGTLVTVSS

SEQ ID NO: 54 - protein, 246 aa, Artificial Sequence
  hBCMA-20 scFv
EIVLTQSPATLSLSPGERATLSCRASESVSVPGISFIHWYQQKPGQAPRLLIYLGRNRAAGIPARFSGSGSGTDFTLTISSLEPEDAAIYYCLQSRRFPRHFGQGTKLEIKGSTSGSGKPGSGEGSTKGQVQLVQSGAEVKKPGASVKVSCKASGYTFADYTINWVRQATGQGLEWMGWINTETREPTYAYDFRSRFTFTLNTSISTAYLELSSLRSEDTAVYYCARDIRYVMDYWGQGTLVTVSS

SEQ ID NO: 55 - protein, 246 aa, Artificial Sequence
  hBCMA-22 scFv
EIVLTQSPATLSLSPGERATLSCRASESVSVPGISFIHWYQQKPGQAPRLLIYLGSNRPSGIPARFSGSGSGTDFTLTISSLEPEDAAIYYCLQSRRFPRHFGQGTKLEIKGSTSGSGKPGSGEGSTKGQVQLVQSGAEVKKPGASVKVSCKASGYTFADYTINWVRQATGQGLEWMGWINTETREPAYAYDFRGRFTFTLNTSISTAYLELSSLRSEDTAVYYCARDIRYVMDYWGQGTLVTVSS

SEQ ID NO: 56 - protein, 246 aa, Artificial Sequence
  hBCMA-23 scFv
EIVLTQSPATLSLSPGERATLSCRASESVSVPGISFIHWYQQKPGQAPRLLIYLGSNRPSGIPARFSGSGSGTDFTLTISSLEPEDAAIYYCLQSRRFPRHFGQGTKLEIKGSTSGSGKPGSGEGSTKGQVQLVQSGAEVKKPGASVKVSCKASGYTFADYTINWVRQATGQGLEWMGWINTETREPTYAYDFRSRFTFTLNTSISTAYLELSSLRSEDTAVYYCARDIRYVMDYWGQGTLVTVSS

SEQ ID NO: 57 - protein, 231 aa, Homo sapiens
  human Fc
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 18 aa, Artificial Sequence
  linker peptide
GSTSGSGKPGSGEGSTKG

SEQ ID NO: 59 - protein, 21 aa, Artificial Sequence
  signal peptide
MALPVTALLLPLALLLHAARP

SEQ ID NO: 60 - protein, 45 aa, Artificial Sequence
  CD8 hinge region
TTTPAPRPPTPAPTIASQPLSLRPEACRPAAGGAVHTRGLDFACD

SEQ ID NO: 61 - protein, 24 aa, Artificial Sequence
  CD8 transmembrane domain
IYIWAPLAGTCGVLLLSLVITLYC

SEQ ID NO: 62 - protein, 42 aa, Artificial Sequence
  CD137 costimulatory domain
KRGRKKLLYIFKQPFMRPVQTTQEEDGCSCRFPEEEEGGCEL

SEQ ID NO: 63 - protein, 112 aa, Artificial Sequence
  CD3zeta intracellular signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 490 aa, Artificial Sequence
  hBCMA-01 CAR
MALPVTALLLPLALLLHAARPEIVLTQSPATLSLSPGERATLSCRASESVSVIGAHLIHWYQQKPGQAPRLLIYLGRNRAAGIPARFSGSGSGTDFTLTISSLEPEDAAIYYCLQSRIFPRTFGQGTKLEIKGSTSGSGKPGSGEGSTKGQVQLVQSGSELKKPGASVKVSCKASGYTFADYTINWVRQATGQGLEWMGWINTETREPAYAYDFRGRFVFSLDTSVSTAYLQISSLKAEDTAVYYCARDIRYVMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 490 aa, Artificial Sequence
  hBCMA-02 CAR
MALPVTALLLPLALLLHAARPEIVLTQSPATLSLSPGERATLSCRASESVSVSGIHLLHWYQQKPGQAPRLLIYLGRNRAAGIPARFSGSGSGTDFTLTISSLEPEDAAIYYCLQSRRFPRHFGQGTKLEIKGSTSGSGKPGSGEGSTKGQVQLVQSGSELKKPGASVKVSCKASGYSFADYTINWVRQAPGQGLEWIGWINTETREPAYAYDFRSRFVFSLDTSASTAYLQISSLKAEDTAVYYCARDIRYVMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - protein, 490 aa, Artificial Sequence
  hBCMA-15 CAR
MALPVTALLLPLALLLHAARPEIVLTQSPATLSLSPGERATLSCRASESVSVSGIHLLHWYQQKPGQAPRLLIYLGRNRAAGIPARFSGSGSGTDFTLTISSLEPEDAAIYYCLQSRRFPRHFGQGTKLEIKGSTSGSGKPGSGEGSTKGQVQLVQSGSEVKQPGASVKVSCKASGYSFADYTINWVRQAPGQGLEWMGWINTETREPTYAYDFRSRFVFSLDTSASTAYLELSSLRSEDTAVYYCARDIRYVMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 490 aa, Artificial Sequence
  hBCMA-17 CAR
MALPVTALLLPLALLLHAARPEIVLTQSPATLSLSPGERATLSCRASESVSVSGIHLLHWYQQKPGQAPRLLIYLGRNRAAGIPARFSGSGSGTDFTLTISSLEPEDAAIYYCLQSRRFPRHFGQGTKLEIKGSTSGSGKPGSGEGSTKGQVQLVQSGAEVKKPGASVKVSCKASGYTFADYTINWVRQATGQGLEWMGWINTETREPTYAYDFRSRFTFTLNTSISTAYLELSSLRSEDTAVYYCARDIRYVMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 490 aa, Artificial Sequence
  hBCMA-19 CAR
MALPVTALLLPLALLLHAARPEIVLTQSPATLSLSPGERATLSCRASESVSVPGISFIHWYQQKPGQAPRLLIYLGRNRAAGIPARFSGSGSGTDFTLTISSLEPEDAAIYYCLQSRRFPRHFGQGTKLEIKGSTSGSGKPGSGEGSTKGQVQLVQSGAEVKKPGASVKVSCKASGYTFADYTINWVRQATGQGLEWMGWINTETREPAYAYDFRGRFTFTLNTSISTAYLELSSLRSEDTAVYYCARDIRYVMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - protein, 490 aa, Artificial Sequence
  hBCMA-20 CAR
MALPVTALLLPLALLLHAARPEIVLTQSPATLSLSPGERATLSCRASESVSVPGISFIHWYQQKPGQAPRLLIYLGRNRAAGIPARFSGSGSGTDFTLTISSLEPEDAAIYYCLQSRRFPRHFGQGTKLEIKGSTSGSGKPGSGEGSTKGQVQLVQSGAEVKKPGASVKVSCKASGYTFADYTINWVRQATGQGLEWMGWINTETREPTYAYDFRSRFTFTLNTSISTAYLELSSLRSEDTAVYYCARDIRYVMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - protein, 490 aa, Artificial Sequence
  hBCMA-22 CAR
MALPVTALLLPLALLLHAARPEIVLTQSPATLSLSPGERATLSCRASESVSVPGISFIHWYQQKPGQAPRLLIYLGSNRPSGIPARFSGSGSGTDFTLTISSLEPEDAAIYYCLQSRRFPRHFGQGTKLEIKGSTSGSGKPGSGEGSTKGQVQLVQSGAEVKKPGASVKVSCKASGYTFADYTINWVRQATGQGLEWMGWINTETREPAYAYDFRGRFTFTLNTSISTAYLELSSLRSEDTAVYYCARDIRYVMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 490 aa, Artificial Sequence
  hBCMA-23 CAR
MALPVTALLLPLALLLHAARPEIVLTQSPATLSLSPGERATLSCRASESVSVPGISFIHWYQQKPGQAPRLLIYLGSNRPSGIPARFSGSGSGTDFTLTISSLEPEDAAIYYCLQSRRFPRHFGQGTKLEIKGSTSGSGKPGSGEGSTKGQVQLVQSGAEVKKPGASVKVSCKASGYTFADYTINWVRQATGQGLEWMGWINTETREPTYAYDFRSRFTFTLNTSISTAYLELSSLRSEDTAVYYCARDIRYVMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - protein, 490 aa, Artificial Sequence
  hBCMA-10 CAR
MALPVTALLLPLALLLHAARPDIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 1098 aa, Artificial Sequence
  huBCMA-10-L6-Li CAR
MALPVTALLLPLALLLHAARPDIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGPMGAVLRSLLACSFCVLLRAEQKLISEEDLEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDELDCYPTEEPAPQATNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDLNYDSEPVPPPPTPRSQYLSAEENYESCPPSPYTERSYSHHLYPPPPSPCTDSSATNFSLLKQAGDVEENPGPMHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCHHHHHH

SEQ ID NO: 74 - protein, 1098 aa, Artificial Sequence
  hBCMA-22-L6-Li CAR
MALPVTALLLPLALLLHAARPEIVLTQSPATLSLSPGERATLSCRASESVSVPGISFIHWYQQKPGQAPRLLIYLGSNRPSGIPARFSGSGSGTDFTLTISSLEPEDAAIYYCLQSRRFPRHFGQGTKLEIKGSTSGSGKPGSGEGSTKGQVQLVQSGAEVKKPGASVKVSCKASGYTFADYTINWVRQATGQGLEWMGWINTETREPAYAYDFRGRFTFTLNTSISTAYLELSSLRSEDTAVYYCARDIRYVMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGPMGAVLRSLLACSFCVLLRAEQKLISEEDLEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDELDCYPTEEPAPQATNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDLNYDSEPVPPPPTPRSQYLSAEENYESCPPSPYTERSYSHHLYPPPPSPCTDSSATNFSLLKQAGDVEENPGPMHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCHHHHHH

SEQ ID NO: 75 - DNA, 333 bp, Artificial Sequence
  VL
gaaatcgttctgacccagagtccagcgacgctgagtctgagtccgggtgaacgcgcgacgctgagttgccgtgccagcgaaagcgttagcgtgattggcgcccatctgatccactggtaccagcagaaaccgggccaagccccacgtctgctgatctatctgggacgcaatcgggcggccggcattccagcccgctttagcggcagcggtagcggtaccgattttacgctgaccatcagcagtctggagccagaagatgccgccatctactactgtctgcagagccgcatcttcccacgcaccttcggccaaggcaccaaactggaaatcaag

SEQ ID NO: 76 - DNA, 333 bp, Artificial Sequence
  VL
gaaatcgttctgacccagagtccagcgacgctgagtctgagtccgggtgaacgcgcgacgctgagttgccgtgccagcgaaagcgttagcgtgagtggcattcacttactccactggtaccagcagaaaccgggccaagccccacgtctgctgatctatctgggacgcaatcgggcggccggcattccagcccgctttagcggcagcggtagcggtaccgattttacgctgaccatcagcagtctggagccagaagatgccgccatctactactgtttacagagcagaaggtttcctcgacacttcggccaaggtaccaaactggaaatcaag

SEQ ID NO: 77 - DNA, 333 bp, Artificial Sequence
  VL
gaaatcgttctgacccagagtccagcgacgctgagtctgagtccgggtgaacgcgcgacgctgagttgccgtgccagcgaaagcgttagcgtgcctggcatatctttcatccactggtaccagcagaaaccgggccaagccccacgtctgctgatctatctgggacgcaatcgggcggccggcattccagcccgctttagcggcagcggtagcggtaccgattttacgctgaccatcagcagtctggagccagaagatgccgccatctactactgtttacagagcagaaggtttcctcgacacttcggccaaggtaccaaactggaaatcaag

SEQ ID NO: 78 - DNA, 333 bp, Artificial Sequence
  VL
gaaatcgttctgacccagagtccagcgacgctgagtctgagtccgggtgaacgcgcgacgctgagttgccgtgccagcgaaagcgttagcgtgcctggcatatctttcatccactggtaccagcagaaaccgggccaagccccacgtctgctgatctatctgggcagcaatcgaccatccggcattccagcccgctttagcggcagcggtagcggtaccgattttacgctgaccatcagcagtctggagccagaagatgccgccatctactactgtttacagagcagaaggtttcctcgacacttcggccaaggcaccaaactggaaatcaag

SEQ ID NO: 79 - DNA, 333 bp, Artificial Sequence
  VL
gacatcgtgctgacccagagccccgcttctttagctgtgtctttaggagagagggccaccatcaattgtcgtgcctccgagtccgtgtccgtgattggcgcccatttaatccactggtaccagcagaaacccggccagccccccaagctgctgatctatttagccagcaatctggagaccggcgtgcccgccagatttagcggaagcggctccggcaccgattttactttaaccatttcctctttacaagctgaggacgccgccatctactactgtttacagagcagaatctttcctcgtaccttcggccaaggtaccaagctggaaatcaag

SEQ ID NO: 80 - DNA, 351 bp, Artificial Sequence
  VH
caagttcagctcgtgcagagcggctccgagctgaagaagcccggcgcttccgtgaaggtgagctgcaaggccagcggatacaccttcgccgattacaccatcaactgggttcgtcaagctaccggtcaaggtctggagtggatgggctggatcaacaccgaaaccagagagcccgcctatgcctacgacttcagaggtcgtttcgtgttctctttagacacaagcgtgagcaccgcctatttacagatcagctctttaaaggccgaggataccgccgtgtactactgcgctcgtgacatcagatacgtgatggactactggggccaaggtacactggtgaccgtgtccagc

SEQ ID NO: 81 - DNA, 351 bp, Artificial Sequence
  VH
caagttcagctcgtgcagagcggctccgagctgaagaagcccggcgcttccgtgaaggtgagctgcaaggccagcggatacagcttcgccgattacaccatcaactgggttcgtcaagctcccggtcaaggtctggagtggataggctggatcaacaccgaaaccagagagcccgcctatgcctacgacttcagaagtcgtttcgtgttctctttagacacaagcgcgagcaccgcctatttacagatcagctctttaaaggccgaggataccgccgtgtactactgcgctcgtgacatcagatacgtgatggactactggggccaaggtacactggtgaccgtgtccagc

SEQ ID NO: 82 - DNA, 351 bp, Artificial Sequence
  VH
caagttcagctcgtgcagagcggctccgaggtgaagcagcccggcgcttccgtgaaggtgagctgcaaggccagcggatacagcttcgccgattacaccatcaactgggttcgtcaagctcccggtcaaggtctggagtggatgggctggatcaacaccgaaaccagagagcccacctatgcctacgacttcagaagtcgtttcgtgttctctttagacacaagcgcgagcaccgcctatttagagctcagctctttaaggtccgaggataccgccgtgtactactgcgctcgtgacatcagatacgtgatggactactggggccaaggtacactggtgaccgtgtccagc

SEQ ID NO: 83 - DNA, 351 bp, Artificial Sequence
  VH
caagttcagctcgtgcagagcggcgccgaggtgaagaagcccggcgcttccgtgaaggtgagctgcaaggccagcggatacaccttcgccgattacaccatcaactgggttcgtcaagctaccggtcaaggtctggagtggatgggctggatcaacaccgaaaccagagagcccacctatgcctacgacttcagaagtcgtttcacgttcactttaaacacaagcatcagcaccgcctatttagagctcagctctttaaggtccgaggataccgccgtgtactactgcgctcgtgacatcagatacgtgatggactactggggccaaggtacactggtgaccgtgtccagc

SEQ ID NO: 84 - DNA, 351 bp, Artificial Sequence
  VH
caagttcagctcgtgcagagcggcgccgaggtgaagaagcccggcgcttccgtgaaggtgagctgcaaggccagcggatacaccttcgccgattacaccatcaactgggttcgtcaagctaccggtcaaggtctggagtggatgggctggatcaacaccgaaaccagagagcccgcctatgcctacgacttcagaggtcgtttcacgttcactttaaacacaagcatcagcaccgcctatttagagctcagctctttaaggtccgaggataccgccgtgtactactgcgctcgtgacatcagatacgtgatggactactggggccaaggtacactggtgaccgtgtccagc

SEQ ID NO: 85 - DNA, 351 bp, Artificial Sequence
  VH
caagttcagctcgtgcagagcggctccgagctgaagaagcccggcgcttccgtgaaggtgagctgcaaggccagcggatacaccttcaccgactactccatcaactgggttcgtcaagctcccggtcaaggtctggagtggatgggctggatcaacaccgaaaccagagagcccgcctatgcctacgacttcagaggtcgtttcgtgttctctttagacacaagcgtgagcaccgcctatttacagatcagctctttaaaggccgaggataccgccgtgtactactgcgctcgtgactacagctacgccatggactactggggccaaggtacactggtgaccgtgtccagc

SEQ ID NO: 86 - DNA, 54 bp, Artificial Sequence
  nucleotides of linker peptides
ggatccaccagcggctccggaaagcccggttccggagagggcagcacaaagggc

SEQ ID NO: 87 - DNA, 51 bp, Artificial Sequence
  forward primer
cctctggctctgctgctgcacgccgcaagacctgaaatcgttctgacccag

SEQ ID NO: 88 - DNA, 58 bp, Artificial Sequence
  reverse primer
tgggcgccggtgttggtggtcgcggcgctggcgtcgtggtgctggacacggtcaccag

SEQ ID NO: 89 - DNA, 735 bp, Artificial Sequence
  signal peptide-CD8 hinge region-CD8 transmembrane domain-CD137 costimulatory domain-CD3zeta intracell ular signaling domain
atggctctgcctgtgaccgctctgctgctgcctctggctctgctgctgcacgccgcaagacctaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 90 - DNA, 63 bp, Artificial Sequence
  signal peptide
atggctctgcctgtgaccgctctgctgctgcctctggctctgctgctgcacgccgcaagacct

SEQ ID NO: 91 - DNA, 135 bp, Artificial Sequence
  CD8 hinge region
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 92 - DNA, 72 bp, Artificial Sequence
  CD8 transmembrane domain
atctacatctgggcgcccttggccgggacttgtggggtccttctcctgtcactggttatcaccctttactgc

SEQ ID NO: 93 - DNA, 126 bp, Artificial Sequence
  CD137 costimulatory domain
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 94 - DNA, 339 bp, Artificial Sequence
  CD3zeta intracellular signaling domain
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 95 - DNA, 1473 bp, Artificial Sequence
  hBCMA-01 CAR
atggctctgcctgtgaccgctctgctgctgcctctggctctgctgctgcacgccgcaagacctgaaatcgttctgacccagagtccagcgacgctgagtctgagtccgggtgaacgcgcgacgctgagttgccgtgccagcgaaagcgttagcgtgattggcgcccatctgatccactggtaccagcagaaaccgggccaagccccacgtctgctgatctatctgggacgcaatcgggcggccggcattccagcccgctttagcggcagcggtagcggtaccgattttacgctgaccatcagcagtctggagccagaagatgccgccatctactactgtctgcagagccgcatcttcccacgcaccttcggccaaggcaccaaactggaaatcaagggatccaccagcggctccggaaagcccggttccggagagggcagcacaaagggccaagttcagctcgtgcagagcggctccgagctgaagaagcccggcgcttccgtgaaggtgagctgcaaggccagcggatacaccttcgccgattacaccatcaactgggttcgtcaagctaccggtcaaggtctggagtggatgggctggatcaacaccgaaaccagagagcccgcctatgcctacgacttcagaggtcgtttcgtgttctctttagacacaagcgtgagcaccgcctatttacagatcagctctttaaaggccgaggataccgccgtgtactactgcgctcgtgacatcagatacgtgatggactactggggccaaggtacactggtgaccgtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 96 - DNA, 1473 bp, Artificial Sequence
  hBCMA-02 CAR
atggctctgcctgtgaccgctctgctgctgcctctggctctgctgctgcacgccgcaagacctgaaatcgttctgacccagagtccagcgacgctgagtctgagtccgggtgaacgcgcgacgctgagttgccgtgccagcgaaagcgttagcgtgagtggcattcacttactccactggtaccagcagaaaccgggccaagccccacgtctgctgatctatctgggacgcaatcgggcggccggcattccagcccgctttagcggcagcggtagcggtaccgattttacgctgaccatcagcagtctggagccagaagatgccgccatctactactgtttacagagcagaaggtttcctcgacacttcggccaaggtaccaaactggaaatcaagggatccaccagcggctccggaaagcccggttccggagagggcagcacaaagggccaagttcagctcgtgcagagcggctccgagctgaagaagcccggcgcttccgtgaaggtgagctgcaaggccagcggatacagcttcgccgattacaccatcaactgggttcgtcaagctcccggtcaaggtctggagtggataggctggatcaacaccgaaaccagagagcccgcctatgcctacgacttcagaagtcgtttcgtgttctctttagacacaagcgcgagcaccgcctatttacagatcagctctttaaaggccgaggataccgccgtgtactactgcgctcgtgacatcagatacgtgatggactactggggccaaggtacactggtgaccgtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 97 - DNA, 1473 bp, Artificial Sequence
  hBCMA-15 CAR
atggctctgcctgtgaccgctctgctgctgcctctggctctgctgctgcacgccgcaagacctgaaatcgttctgacccagagtccagcgacgctgagtctgagtccgggtgaacgcgcgacgctgagttgccgtgccagcgaaagcgttagcgtgagtggcattcacttactccactggtaccagcagaaaccgggccaagccccacgtctgctgatctatctgggacgcaatcgggcggccggcattccagcccgctttagcggcagcggtagcggtaccgattttacgctgaccatcagcagtctggagccagaagatgccgccatctactactgtttacagagcagaaggtttcctcgacacttcggccaaggtaccaaactggaaatcaagggatccaccagcggctccggaaagcccggttccggagagggcagcacaaagggccaagttcagctcgtgcagagcggctccgaggtgaagcagcccggcgcttccgtgaaggtgagctgcaaggccagcggatacagcttcgccgattacaccatcaactgggttcgtcaagctcccggtcaaggtctggagtggatgggctggatcaacaccgaaaccagagagcccacctatgcctacgacttcagaagtcgtttcgtgttctctttagacacaagcgcgagcaccgcctatttagagctcagctctttaaggtccgaggataccgccgtgtactactgcgctcgtgacatcagatacgtgatggactactggggccaaggtacactggtgaccgtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 98 - DNA, 1473 bp, Artificial Sequence
  hBCMA-17 CAR
atggctctgcctgtgaccgctctgctgctgcctctggctctgctgctgcacgccgcaagacctgaaatcgttctgacccagagtccagcgacgctgagtctgagtccgggtgaacgcgcgacgctgagttgccgtgccagcgaaagcgttagcgtgagtggcattcacttactccactggtaccagcagaaaccgggccaagccccacgtctgctgatctatctgggacgcaatcgggcggccggcattccagcccgctttagcggcagcggtagcggtaccgattttacgctgaccatcagcagtctggagccagaagatgccgccatctactactgtttacagagcagaaggtttcctcgacacttcggccaaggtaccaaactggaaatcaagggatccaccagcggctccggaaagcccggttccggagagggcagcacaaagggccaagttcagctcgtgcagagcggcgccgaggtgaagaagcccggcgcttccgtgaaggtgagctgcaaggccagcggatacaccttcgccgattacaccatcaactgggttcgtcaagctaccggtcaaggtctggagtggatgggctggatcaacaccgaaaccagagagcccacctatgcctacgacttcagaagtcgtttcacgttcactttaaacacaagcatcagcaccgcctatttagagctcagctctttaaggtccgaggataccgccgtgtactactgcgctcgtgacatcagatacgtgatggactactggggccaaggtacactggtgaccgtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 99 - DNA, 1473 bp, Artificial Sequence
  hBCMA-19 CAR
atggctctgcctgtgaccgctctgctgctgcctctggctctgctgctgcacgccgcaagacctgaaatcgttctgacccagagtccagcgacgctgagtctgagtccgggtgaacgcgcgacgctgagttgccgtgccagcgaaagcgttagcgtgcctggcatatctttcatccactggtaccagcagaaaccgggccaagccccacgtctgctgatctatctgggacgcaatcgggcggccggcattccagcccgctttagcggcagcggtagcggtaccgattttacgctgaccatcagcagtctggagccagaagatgccgccatctactactgtttacagagcagaaggtttcctcgacacttcggccaaggtaccaaactggaaatcaagggatccaccagcggctccggaaagcccggttccggagagggcagcacaaagggccaagttcagctcgtgcagagcggcgccgaggtgaagaagcccggcgcttccgtgaaggtgagctgcaaggccagcggatacaccttcgccgattacaccatcaactgggttcgtcaagctaccggtcaaggtctggagtggatgggctggatcaacaccgaaaccagagagcccgcctatgcctacgacttcagaggtcgtttcacgttcactttaaacacaagcatcagcaccgcctatttagagctcagctctttaaggtccgaggataccgccgtgtactactgcgctcgtgacatcagatacgtgatggactactggggccaaggtacactggtgaccgtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 100 - DNA, 1473 bp, Artificial Sequence
  hBCMA-20 CAR
atggctctgcctgtgaccgctctgctgctgcctctggctctgctgctgcacgccgcaagacctgaaatcgttctgacccagagtccagcgacgctgagtctgagtccgggtgaacgcgcgacgctgagttgccgtgccagcgaaagcgttagcgtgcctggcatatctttcatccactggtaccagcagaaaccgggccaagccccacgtctgctgatctatctgggacgcaatcgggcggccggcattccagcccgctttagcggcagcggtagcggtaccgattttacgctgaccatcagcagtctggagccagaagatgccgccatctactactgtttacagagcagaaggtttcctcgacacttcggccaaggtaccaaactggaaatcaagggatccaccagcggctccggaaagcccggttccggagagggcagcacaaagggccaagttcagctcgtgcagagcggcgccgaggtgaagaagcccggcgcttccgtgaaggtgagctgcaaggccagcggatacaccttcgccgattacaccatcaactgggttcgtcaagctaccggtcaaggtctggagtggatgggctggatcaacaccgaaaccagagagcccacctatgcctacgacttcagaagtcgtttcacgttcactttaaacacaagcatcagcaccgcctatttagagctcagctctttaaggtccgaggataccgccgtgtactactgcgctcgtgacatcagatacgtgatggactactggggccaaggtacactggtgaccgtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 101 - DNA, 1473 bp, Artificial Sequence
  hBCMA-22 CAR
atggctctgcctgtgaccgctctgctgctgcctctggctctgctgctgcacgccgcaagacctgaaatcgttctgacccagagtccagcgacgctgagtctgagtccgggtgaacgcgcgacgctgagttgccgtgccagcgaaagcgttagcgtgcctggcatatctttcatccactggtaccagcagaaaccgggccaagccccacgtctgctgatctatctgggcagcaatcgaccatccggcattccagcccgctttagcggcagcggtagcggtaccgattttacgctgaccatcagcagtctggagccagaagatgccgccatctactactgtttacagagcagaaggtttcctcgacacttcggccaaggcaccaaactggaaatcaagggatccaccagcggctccggaaagcccggttccggagagggcagcacaaagggccaagttcagctcgtgcagagcggcgccgaggtgaagaagcccggcgcttccgtgaaggtgagctgcaaggccagcggatacaccttcgccgattacaccatcaactgggttcgtcaagctaccggtcaaggtctggagtggatgggctggatcaacaccgaaaccagagagcccgcctatgcctacgacttcagaggtcgtttcacgttcactttaaacacaagcatcagcaccgcctatttagagctcagctctttaaggtccgaggataccgccgtgtactactgcgctcgtgacatcagatacgtgatggactactggggccaaggtacactggtgaccgtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 102 - DNA, 1473 bp, Artificial Sequence
  hBCMA-23 CAR
atggctctgcctgtgaccgctctgctgctgcctctggctctgctgctgcacgccgcaagacctgaaatcgttctgacccagagtccagcgacgctgagtctgagtccgggtgaacgcgcgacgctgagttgccgtgccagcgaaagcgttagcgtgcctggcatatctttcatccactggtaccagcagaaaccgggccaagccccacgtctgctgatctatctgggcagcaatcgaccatccggcattccagcccgctttagcggcagcggtagcggtaccgattttacgctgaccatcagcagtctggagccagaagatgccgccatctactactgtttacagagcagaaggtttcctcgacacttcggccaaggcaccaaactggaaatcaagggatccaccagcggctccggaaagcccggttccggagagggcagcacaaagggccaagttcagctcgtgcagagcggcgccgaggtgaagaagcccggcgcttccgtgaaggtgagctgcaaggccagcggatacaccttcgccgattacaccatcaactgggttcgtcaagctaccggtcaaggtctggagtggatgggctggatcaacaccgaaaccagagagcccacctatgcctacgacttcagaagtcgtttcacgttcactttaaacacaagcatcagcaccgcctatttagagctcagctctttaaggtccgaggataccgccgtgtactactgcgctcgtgacatcagatacgtgatggactactggggccaaggtacactggtgaccgtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 103 - DNA, 1473 bp, Artificial Sequence
  hBCMA-10 CAR
atggctctgcctgtgaccgctctgctgctgcctctggctctgctgctgcacgccgcaagacctgacatcgtgctgacccagagccccgcttctttagctgtgtctttaggagagagggccaccatcaattgtcgtgcctccgagtccgtgtccgtgattggcgcccatttaatccactggtaccagcagaaacccggccagccccccaagctgctgatctatttagccagcaatctggagaccggcgtgcccgccagatttagcggaagcggctccggcaccgattttactttaaccatttcctctttacaagctgaggacgccgccatctactactgtttacagagcagaatctttcctcgtaccttcggccaaggtaccaagctggaaatcaagggatccaccagcggctccggaaagcccggttccggagagggcagcacaaagggccaagttcagctcgtgcagagcggctccgagctgaagaagcccggcgcttccgtgaaggtgagctgcaaggccagcggatacaccttcaccgactactccatcaactgggttcgtcaagctcccggtcaaggtctggagtggatgggctggatcaacaccgaaaccagagagcccgcctatgcctacgacttcagaggtcgtttcgtgttctctttagacacaagcgtgagcaccgcctatttacagatcagctctttaaaggccgaggataccgccgtgtactactgcgctcgtgactacagctacgccatggactactggggccaaggtacactggtgaccgtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 104 - DNA, 3294 bp, Artificial Sequence
  huBCMA-10-L6-Li CAR
atggctctgcctgtgaccgctctgctgctgcctctggctctgctgctgcacgccgcaagacctgacatcgtgctgacccagagccccgcttctttagctgtgtctttaggagagagggccaccatcaattgtcgtgcctccgagtccgtgtccgtgattggcgcccatttaatccactggtaccagcagaaacccggccagccccccaagctgctgatctatttagccagcaatctggagaccggcgtgcccgccagatttagcggaagcggctccggcaccgattttactttaaccatttcctctttacaagctgaggacgccgccatctactactgtttacagagcagaatctttcctcgtaccttcggccaaggtaccaagctggaaatcaagggatccaccagcggctccggaaagcccggttccggagagggcagcacaaagggccaagttcagctcgtgcagagcggctccgagctgaagaagcccggcgcttccgtgaaggtgagctgcaaggccagcggatacaccttcaccgactactccatcaactgggttcgtcaagctcccggtcaaggtctggagtggatgggctggatcaacaccgaaaccagagagcccgcctatgcctacgacttcagaggtcgtttcgtgttctctttagacacaagcgtgagcaccgcctatttacagatcagctctttaaaggccgaggataccgccgtgtactactgcgctcgtgactacagctacgccatggactactggggccaaggtacactggtgaccgtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgagggcagaggaagtcttctaacatgcggtgacgtggaggagaatcccggccctatgggggccgtcctgaggagcctcctggcctgcagcttctgtgtgctcctgagagcggagcagaaactcatctctgaagaggatctggaacctccaacatgttctcctcagcagtttacttgtttcacgggggaaattgactgtatccctgtggcttggcggtgcgatgggtttactgaatgtgaagaccacagtgatgaactcaattgtcctgtatgctcagagtcccagttccagtgtgccagtgggcagtgtattgatggtgccctccgatgcaatggagatgcaaactgccaggacaaatcagatgagaagaactgtgaagtgctttgtttaattgatcagttccgctgtgccaatggtcagtgcattggaaagcacaagaagtgtgatcataatgtggattgcagtgacaagtcagatgaactggattgttatccgactgaagaaccagcaccacaggccaccaatacagttggttctgttattggcgtaattgtcaccatttttgtgtctggaactgtatactttatctgccagaggatgttgtgtccacgtatgaagggagatggggaaactatgactaatgactatgtagttcatggaccagcttctgtgcctcttggttatgtgccacacccaagttctttgtcaggatctcttccaggaatgtctcgaggtaaatcaatgatcagctccctcagtatcatggggggaagcagtggacccccctatgaccgagcccatgttacaggagcatcatcaagtagttcttcaagcaccaaaggcacttacttccctgcaattttgaaccctccaccatccccagccacagagcgatcacattacactatggaatttggatattcttcaaacagtccttccactcataggtcatacagctacaggccatatagctaccggcactttgcaccccccaccacaccctgcagcacagatgtttgtgacagtgactatgctcctagtcggagaatgacctcagtggcaacagccaagggctataccagtgacttgaactatgattcagaacctgtgcccccacctcccacaccccgaagccaatacttgtcagcagaggagaactatgaaagctgcccaccttctccatacacagagaggagctattctcatcacctctacccaccgccaccctctccctgtacagactcctccgccaccaattttagcctgctgaaacaggccggagacgtggaggaaaaccccggccccatgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgccaccatcaccaccatcac

SEQ ID NO: 105 - DNA, 3294 bp, Artificial Sequence
  hBCMA-22-L6-Li CAR
atggctctgcctgtgaccgctctgctgctgcctctggctctgctgctgcacgccgcaagacctgaaatcgttctgacccagagtccagcgacgctgagtctgagtccgggtgaacgcgcgacgctgagttgccgtgccagcgaaagcgttagcgtgcctggcatatctttcatccactggtaccagcagaaaccgggccaagccccacgtctgctgatctatctgggcagcaatcgaccatccggcattccagcccgctttagcggcagcggtagcggtaccgattttacgctgaccatcagcagtctggagccagaagatgccgccatctactactgtttacagagcagaaggtttcctcgacacttcggccaaggcaccaaactggaaatcaagggatccaccagcggctccggaaagcccggttccggagagggcagcacaaagggccaagttcagctcgtgcagagcggcgccgaggtgaagaagcccggcgcttccgtgaaggtgagctgcaaggccagcggatacaccttcgccgattacaccatcaactgggttcgtcaagctaccggtcaaggtctggagtggatgggctggatcaacaccgaaaccagagagcccgcctatgcctacgacttcagaggtcgtttcacgttcactttaaacacaagcatcagcaccgcctatttagagctcagctctttaaggtccgaggataccgccgtgtactactgcgctcgtgacatcagatacgtgatggactactggggccaaggtacactggtgaccgtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgagggcagaggaagtcttctaacatgcggtgacgtggaggagaatcccggccctatgggggccgtcctgaggagcctcctggcctgcagcttctgtgtgctcctgagagcggagcagaaactcatctctgaagaggatctggaacctccaacatgttctcctcagcagtttacttgtttcacgggggaaattgactgtatccctgtggcttggcggtgcgatgggtttactgaatgtgaagaccacagtgatgaactcaattgtcctgtatgctcagagtcccagttccagtgtgccagtgggcagtgtattgatggtgccctccgatgcaatggagatgcaaactgccaggacaaatcagatgagaagaactgtgaagtgctttgtttaattgatcagttccgctgtgccaatggtcagtgcattggaaagcacaagaagtgtgatcataatgtggattgcagtgacaagtcagatgaactggattgttatccgactgaagaaccagcaccacaggccaccaatacagttggttctgttattggcgtaattgtcaccatttttgtgtctggaactgtatactttatctgccagaggatgttgtgtccacgtatgaagggagatggggaaactatgactaatgactatgtagttcatggaccagcttctgtgcctcttggttatgtgccacacccaagttctttgtcaggatctcttccaggaatgtctcgaggtaaatcaatgatcagctccctcagtatcatggggggaagcagtggacccccctatgaccgagcccatgttacaggagcatcatcaagtagttcttcaagcaccaaaggcacttacttccctgcaattttgaaccctccaccatccccagccacagagcgatcacattacactatggaatttggatattcttcaaacagtccttccactcataggtcatacagctacaggccatatagctaccggcactttgcaccccccaccacaccctgcagcacagatgtttgtgacagtgactatgctcctagtcggagaatgacctcagtggcaacagccaagggctataccagtgacttgaactatgattcagaacctgtgcccccacctcccacaccccgaagccaatacttgtcagcagaggagaactatgaaagctgcccaccttctccatacacagagaggagctattctcatcacctctacccaccgccaccctctccctgtacagactcctccgccaccaattttagcctgctgaaacaggccggagacgtggaggaaaaccccggccccatgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgccaccatcaccaccatcac

SEQ ID NO: 106 - DNA, 1824 bp, Artificial Sequence
  L6-Li nucleotide sequence
gagggcagaggaagtcttctaacatgcggtgacgtggaggagaatcccggccctatgggggccgtcctgaggagcctcctggcctgcagcttctgtgtgctcctgagagcggagcagaaactcatctctgaagaggatctggaacctccaacatgttctcctcagcagtttacttgtttcacgggggaaattgactgtatccctgtggcttggcggtgcgatgggtttactgaatgtgaagaccacagtgatgaactcaattgtcctgtatgctcagagtcccagttccagtgtgccagtgggcagtgtattgatggtgccctccgatgcaatggagatgcaaactgccaggacaaatcagatgagaagaactgtgaagtgctttgtttaattgatcagttccgctgtgccaatggtcagtgcattggaaagcacaagaagtgtgatcataatgtggattgcagtgacaagtcagatgaactggattgttatccgactgaagaaccagcaccacaggccaccaatacagttggttctgttattggcgtaattgtcaccatttttgtgtctggaactgtatactttatctgccagaggatgttgtgtccacgtatgaagggagatggggaaactatgactaatgactatgtagttcatggaccagcttctgtgcctcttggttatgtgccacacccaagttctttgtcaggatctcttccaggaatgtctcgaggtaaatcaatgatcagctccctcagtatcatggggggaagcagtggacccccctatgaccgagcccatgttacaggagcatcatcaagtagttcttcaagcaccaaaggcacttacttccctgcaattttgaaccctccaccatccccagccacagagcgatcacattacactatggaatttggatattcttcaaacagtccttccactcataggtcatacagctacaggccatatagctaccggcactttgcaccccccaccacaccctgcagcacagatgtttgtgacagtgactatgctcctagtcggagaatgacctcagtggcaacagccaagggctataccagtgacttgaactatgattcagaacctgtgcccccacctcccacaccccgaagccaatacttgtcagcagaggagaactatgaaagctgcccaccttctccatacacagagaggagctattctcatcacctctacccaccgccaccctctccctgtacagactcctccgccaccaattttagcctgctgaaacaggccggagacgtggaggaaaaccccggccccatgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgccaccatcaccaccatcac

SEQ ID NO: 107 - protein, 608 aa, Artificial Sequence
  L6-Li amino acid sequence
EGRGSLLTCGDVEENPGPMGAVLRSLLACSFCVLLRAEQKLISEEDLEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDELDCYPTEEPAPQATNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDLNYDSEPVPPPPTPRSQYLSAEENYESCPPSPYTERSYSHHLYPPPPSPCTDSSATNFSLLKQAGDVEENPGPMHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCHHHHHH